(19) United States
(12) Patent Application Publication
(10) Pub. No.: US 2018/0160921 A1
(43) Pub. Date: Jun. 14, 2018

(54) PHYSICAL ABILITY EVALUATION SYSTEM, ELECTRONIC APPARATUS, PHYSICAL ABILITY EVALUATION SERVER, PHYSICAL ABILITY EVALUATION METHOD, PHYSICAL ABILITY EVALUATION PROGRAM, AND RECORDING MEDIUM

(71) Applicant: SEIKO EPSON CORPORATION, Tokyo (JP)

(72) Inventor: Tsubasa SHIRAI, Shiojiri-shi (JP)

(73) Assignee: SEIKO EPSON CORPORATION, Tokyo (JP)

(21) Appl. No.: 15/833,373

(22) Filed: Dec. 6, 2017

(30) Foreign Application Priority Data
Dec. 13, 2016 (JP) 2016-241622

(51) Int. Cl. A61B 5/024; A61B 5/00; A61B 5/0255

(52) U.S. Cl. CPC A61B 5/02405; A61B 5/02438; A61B 5/743; A61B 5/0022; A61B 5/486; A61B 5/681; A61B 5/7445; A61B 5/0255

(57) ABSTRACT

A physical ability evaluation system includes an output unit that outputs a plurality of indexes related to heart beat information of a user as information related to balance of a physical ability of the user.

### CROSS-REFERENCES TO RELATED APPLICATIONS

This application claims priority to Japanese Patent Application No. 2016-241622, filed Dec. 13, 2016, the entirety of which is herein incorporated by reference.

### BACKGROUND

### 1. Technical Field

The present invention relates to a physical ability evaluation system, an electronic apparatus, a physical ability evaluation server, a physical ability evaluation method, a physical ability evaluation program, and a recording medium.

### 2. Related Art

JP-A-2015-210116 discloses a Global Positioning System (GPS) device that supplies information having an influence on a necessary time of a mountaineering route to a user based on a movement history of the user and a movement history of another user. JP-A-2015-210116 discloses a system that predicts a goal arrival time by uploading course information and pace information of a user to a server and statistically comparing the course information and the pace information to previous data of a plurality of users.

However, since information to be supplied to users by the system is simple statistical data which is based on data of many unspecified users, the information is not necessarily effective information for all the users. For example, the information is not sufficient for users such as athletes who have excellent physical abilities, users who have purposes of executing training, and users who feel uneasy about health.

### SUMMARY

An advantage of some aspects of the invention is that it provides a physical ability evaluation system, an electronic apparatus, a physical ability evaluation server, a physical ability evaluation method, a physical ability evaluation program, and a recording medium capable of objectively and simply evaluating a physical ability type of an individual user who is exercising.

The invention can be implemented as the following forms or application examples.

### APPLICATION EXAMPLE 1

A physical ability evaluation system according to this application example includes an output unit that outputs a plurality of indexes related to heart beat information of a user as information related to balance of a physical ability of the user.

In the related art, a user merely experimentally grasped to which type the user is relevant among various types such as a type strong in a long distance, a type strong to a short distance, a type strong an uphill ground, a type strong to a downhill ground, and a type strong to a flat ground. However, according to the information (that is, the information related to the balance of the physical ability) which is based on the plurality of indexes related to the heart beat information, the user can objectively grasp his or her physical ability type.

### APPLICATION EXAMPLE 2

In the physical ability evaluation system according to the application example, the plurality of indexes may include at least one of an index indicating a degree of a change in the heart beat information with respect to an elapsed time of the user, an index indicating a degree of a change in the heart beat information with respect to a change in a speed of the user in a horizontal direction perpendicular to a gravity direction, and an index indicating a degree of a change in the heart beat information with respect to a speed of the user the gravity direction.

An energy consumption rate of the user during exercise or stop is reflected in the index which is based on the elapsed time (a stamina index to be described below) among the plurality of indexes. Therefore, in particular, the energy consumption rate among individual user physical abilities can be evaluated objectively and simply with the index (the energy consumption rate mentioned here is assumed to include meaning of an energy recovery rate and is also referred to as “stamina”).

Conversion efficiency from beating of the heart of the user who is exercising to a movement speed in the horizontal direction is reflected in the index (corresponding to a gear index to be described below) which is based on the change in the speed in the horizontal direction among the plurality of indexes. Therefore, in particular, horizontal movement efficiency can be evaluated objectively and simply with the index among the individual user physical abilities.

Conversion efficiency from beating of the heart of the user who is exercising to a movement speed in the gravity direction is reflected in the index (a slope ground adaptability index to be described below) which is a based on the change in the speed in the gravity direction among the plurality of indexes. Therefore, in particular, movement efficiency in the gravity direction can be evaluated objectively and simply among the individual user physical abilities.

The “heart beat information” includes information related to the number of cardiac beats per unit time, such as information regarding a heart rate, information regarding a pulse rate, and electrocardiographic information. The output unit can be configured with a processing unit, a display unit, a sound output unit, a communication unit, or the like. An output destination of the index may be a user or a device.

### APPLICATION EXAMPLE 3

In the physical ability evaluation system according to the application example, the plurality of indexes may include at least of a plurality of indexes calculated for each period in which the user exercises, a plurality of indexes calculated for each weather of a route along which the user exercises, and a plurality of indexes calculated for each gradient of the route along which the user exercises.

Accordingly, it is possible to make any comparison among comparison by a period of the balance of the physical ability, comparison by the weather, and comparison by the gradient.

The comparison by the period is, for example, comparison between the balances at temporally different previous times or comparison between the balances at previous and current times. The “current time” may include a given period of time including the current time.

The comparison by the weather is, for example, comparison by a difference among a fair sky, a rainy sky, a cloudy sky or comparison by atmospheric pressure. In this case, it is possible to analyze a change in the balance which depends on weather of the route of the exercise.

The comparison by the gradient is, for example, comparison by a difference among an uphill ground, a flat ground, and a downhill ground. In this case, it is possible to analyze a change in the balance which depends on the gradient of the route of the exercise.

### APPLICATION EXAMPLE 4

In the physical ability evaluation system according to the application example, the output unit may output a graph related to the plurality of indexes.

According to the graph, it is easy to intuitively grasp the balance of the physical ability.

### APPLICATION EXAMPLE 5

In the physical ability evaluation system according to the application example, the number of users may be plural. The output units may output the graph related to the plurality of users.

According to the graph, it is easy to compare balances of the physical abilities of the plurality of users to each other.

### APPLICATION EXAMPLE 6

In the physical ability evaluation system according to the application example, the plurality of users may be users belonging to the same group.

With this configuration, it is easy to compare the users belonging to the same group. The same group is, for example, a group of users who all execute an exercise. For example, the same group is a mountaineering party moving together along the same route or a long-distance race team running along different sections of the same route in sequence.

### APPLICATION EXAMPLE 7

In the physical ability evaluation system according to the application example, the output unit may output a diagnosis result for each plurality of users.

With this configuration, it is possible to evaluate the plurality of users objectively and fairly.

### APPLICATION EXAMPLE 8

In the physical ability evaluation system according to the application example, the diagnosis result may include at least one of a physical ability type of the user and advice for the user.

With this configuration, it is easy to plan content (route selection, role-sharing, and the like) of exercise of the plurality of users or achieve an improvement in safety or performance of all the plurality of users.

### APPLICATION EXAMPLE 9

The physical ability evaluation system according to the application example may further include a storage unit that stores route information of a route along which the user exercises and the heart beat information in association with each other.

The storage unit can manage the heart beat information in a state associated with the route information. When the heart beat information which is an index calculation source is stored in the storage unit, the heart beat information can also be used for other usages as necessary.

### APPLICATION EXAMPLE 10

An electronic apparatus according to this application example includes an output unit that outputs a plurality of indexes related to heart beat information of a user as information related to balance of a physical ability of the user.

In the related art, a user experimentally grasped to which type the user is relevant among various types such as a type strong in a long distance, a type strong to a short distance, a type strong an uphill ground, a type strong to a downhill ground, and a type strong to a flat ground. In contrast, according to the information (that is, the information related to the balance of the physical ability) which is based on the plurality of indexes related to the heart beat information, the user can objectively grasp his or her physical ability type.

### APPLICATION EXAMPLE 11

A physical ability evaluation server according to this application example includes an output unit that outputs a plurality of indexes related to heart beat information of a user as information related to balance of a physical ability of the user.

In the related art, a user experimentally grasped to which type the user is relevant among various types such as a type strong in a long distance, a type strong to a short distance, a type strong an uphill ground, a type strong to a downhill ground, and a type strong to a flat ground. In contrast, according to the information (that is, the information related to the balance of the physical ability) which is based on the plurality of indexes related to the heart beat information, the user can objectively grasp his or her physical ability type.

### APPLICATION EXAMPLE 12

A physical ability evaluation method according to this application example includes outputting a plurality of indexes related to heart beat information of a user as information related to balance of a physical ability of the user.

In the related art, a user experimentally grasped to which type the user is relevant among various types such as a type strong in a long distance, a type strong to a short distance, a type strong an uphill ground, a type strong to a downhill ground, and a type strong to a flat ground. In contrast, according to the information (that is, the information related to the balance of the physical ability) which is based on the plurality of indexes related to the heart beat information, the user can objectively grasp his or her physical ability type.

### APPLICATION EXAMPLE 13

In the physical ability evaluation method according to the application example, the plurality of indexes may include at least one of an index indicating a degree of a change in the heart beat information with respect to an elapsed time of the user, an index indicating a degree of a change in the heart beat information with respect to a change in a speed of the user in a horizontal direction perpendicular to a gravity direction, and an index indicating a degree of a change in the heart beat information with respect to a speed of the user the gravity direction.

An energy consumption rate of the user during exercise or stop is reflected in the index which is based on the elapsed time (a stamina index to be described below) among the plurality of indexes. Therefore, in particular, the energy consumption rate among individual user physical abilities can be evaluated objectively and simply with the index (the energy consumption rate mentioned here is assumed to include meaning of an energy recovery rate and is also referred to as “stamina”).

Conversion efficiency from beating of the heart of the user who is exercising to a movement speed in the horizontal direction is reflected in the index (corresponding to a gear index to be described below) which is based on the change in the speed in the horizontal direction among the plurality of indexes. Therefore, in particular, horizontal movement efficiency can be evaluated objectively and simply among the individual user physical abilities.

Conversion efficiency from beating of the heart of the user who is exercising to a movement speed in the gravity direction is reflected in the index (a slope ground adaptability index to be described below) which is a based on the change in the speed in the gravity direction among the plurality of indexes. Therefore, in particular, movement efficiency in the gravity direction can be evaluated objectively and simply among the individual user physical abilities.

The “heart beat information” includes information related to the number of cardiac beats per unit time, such as information regarding a heart rate, information regarding a pulse rate, and electrocardiographic information. The output unit can be configured with a processing unit, a display unit, a sound output unit, a communication unit, or the like. An output destination of the index may be a user or a device.

### APPLICATION EXAMPLE 14

In the physical ability evaluation method according to the application example, the plurality of indexes may include at least of a plurality of indexes calculated for each period in which the user exercises, a plurality of indexes calculated for each weather of a route along which the user exercises, and a plurality of indexes calculated for each gradient of the route along which the user exercises.

With this configuration, it is possible to make any comparison among comparison by a period of the balance of the physical ability, comparison by the weather, and comparison by the gradient.

The comparison by the period is, for example, comparison between the plurality of balances at temporally different previous times or comparison between the balances at previous and current times. The “current time” mentioned here may include a given period of time including the current time.

The comparison by the weather is, for example, comparison by a difference among a fair sky, a rainy sky, a cloudy sky or comparison by atmospheric pressure. In this case, it is possible to analyze a change in the balance which depends on weather of the route of the exercise.

The comparison by the gradient is, for example, comparison by a difference among an uphill ground, a flat ground, and a downhill ground. In this case, it is possible to analyze a change in the balance which depends on the gradient of the route of the exercise.

### APPLICATION EXAMPLE 15

In the physical ability evaluation method according to the application example, in the outputting of the plurality of indexes, a graph related to the plurality of indexes may be output.

According to the graph, it is easy to intuitively grasp the balance of the physical ability.

### APPLICATION EXAMPLE 16

In the physical ability evaluation method according to the application example, the number of users may be plural. In the outputting of the plurality of indexes, the graph related to the plurality of users may be output.

According to the graph, it is easy to compare balances of the physical abilities of the plurality of users to each other.

### APPLICATION EXAMPLE 17

In the physical ability evaluation method according to the application example, the plurality of users may be users belonging to the same group.

With this configuration, it is easy to compare the users belonging to the same group. The same group is, for example, a group of users who all execute an exercise. For example, the same group is a mountaineering party moving together along the same route or a long-distance race team running along different sections of the same route in sequence.

### APPLICATION EXAMPLE 18

In the physical ability evaluation method according to the application example, in the outputting of the plurality of indexes, a diagnosis result for each plurality of users may be output.

With this configuration, it is possible to evaluate the plurality of users objectively and fairly.

### APPLICATION EXAMPLE 19

In the physical ability evaluation method according to the application example, the diagnosis result may include at least one of a physical ability type of the user and advice for the user.

With this configuration, it is easy to plan content (route selection, role-sharing, and the like) of exercise of the plurality of users or achieve an improvement in safety or performance of all the plurality of users.

### APPLICATION EXAMPLE 20

A physical ability evaluation program according to this application example causes a computer to perform outputting a plurality of indexes related to heart beat information of a user as information related to balance of a physical ability of the user.

In the related art, a user experimentally grasped to which type the user is relevant among various types such as a type strong in a long distance, a type strong to a short distance, a type strong an uphill ground, a type strong to a downhill ground, and a type strong to a flat ground. However, according to the information (that is, the information related to the balance of the physical ability) which is based on the plurality of indexes related to the heart beat information, the user can objectively grasp his or her physical ability type.

### APPLICATION EXAMPLE 21

A recording medium according to this application example records a program causing a computer to perform outputting a plurality of indexes related to heart beat information of a user as information related to balance of a physical ability of the user.

In the related art, a user experimentally grasped to which type the user is relevant among various types such as a type strong in a long distance, a type strong to a short distance, a type strong an uphill ground, a type strong to a downhill ground, and a type strong to a flat ground. In contrast, according to the information (that is, the information related to the balance of the physical ability) which is based on the plurality of indexes related to the heart beat information, the user can objectively grasp his or her physical ability type.

### APPLICATION EXAMPLE 22

An electronic apparatus according to this application example outputs a plurality of indexes related to heart beat information of a user as information related to balance of a physical ability of the user.

In the related art, a user experimentally grasped to which type the user is relevant among various types such as a type strong in a long distance, a type strong to a short distance, a type strong an uphill ground, a type strong to a downhill ground, and a type strong to a flat ground. In contrast, according to the information (that is, the information related to the balance of the physical ability) which is based on the plurality of indexes related to the heart beat information, the user can objectively grasp his or her physical ability type.

### APPLICATION EXAMPLE 23

In the electronic apparatus according to the application example, the plurality of indexes may include at least one of an index indicating a degree of a change in the heart beat information with respect to an elapsed time of the user, an index indicating a degree of a change in the heart beat information with respect to a change in a speed of the user in a horizontal direction perpendicular to a gravity direction, and an index indicating a degree of a change in the heart beat information with respect to a speed of the user the gravity direction.

An energy consumption rate of the user during exercise or stop is reflected in the index which is based on the elapsed time (a stamina index to be described below) among the plurality of indexes. Therefore, in particular, the energy consumption rate among individual user physical abilities can be evaluated objectively and simply with the index (the energy consumption rate mentioned here is assumed to include meaning of an energy recovery rate and is also referred to as “stamina”).

Conversion efficiency from beating of the heart of the user who is exercising to a movement speed in the horizontal direction is reflected in the index (corresponding to a gear index to be described below) which is based on the change in the speed in the horizontal direction among the plurality of indexes. Therefore, in particular, horizontal movement efficiency can be evaluated objectively and simply among the individual user physical abilities.

Conversion efficiency from beating of the heart of the user who is exercising to a movement speed in the gravity direction is reflected in the index (a slope ground adaptability index to be described below) which is a based on the change in the speed in the gravity direction among the plurality of indexes. Therefore, in particular, movement efficiency in the gravity direction can be evaluated objectively and simply among the individual user physical abilities.

The “heart beat information” includes information related to the number of cardiac beats per unit time, such as information regarding a heart rate, information regarding a pulse rate, and electrocardiographic information. The output unit can be configured with a processing unit, a display unit, a sound output unit, a communication unit, or the like. An output destination of the index may be a user or a device.

### APPLICATION EXAMPLE 24

In the electronic apparatus according to the application example, the plurality of indexes may include at least of a plurality of indexes calculated for each period in which the user exercises, a plurality of indexes calculated for each weather of a route along which the user exercises, and a plurality of indexes calculated for each gradient of the route along which the user exercises.

With this configuration, it is possible to make any comparison among comparison by a period of the balance of the physical ability, comparison by the weather, and comparison by the gradient.

The comparison by the period is, for example, comparison between the plurality of balances at temporally different previous times or comparison between the balances at previous and current times. The “current time” mentioned here may include a given period of time including the current time.

The comparison by the weather is, for example, comparison by a difference among a fair sky, a rainy sky, a cloudy sky or comparison by atmospheric pressure. In this case, it is possible to analyze a change in the balance which depends on weather of the route of the exercise.

The comparison by the gradient is, for example, comparison by an uphill ground, a flat ground, and a downhill ground. In this case, it is possible to analyze a change in the balance which depends on the gradient of the route of the exercise.

### APPLICATION EXAMPLE 25

In the electronic apparatus according to the application example, a graph related to the plurality of indexes may be output.

According to the graph, it is easy to intuitively grasp the balance of the physical ability.

### APPLICATION EXAMPLE 26

In the electronic apparatus according to the application example, the number of users may be plural. The graph related to the plurality of users may be output.

According to the graph, it is easy to compare balances of the physical abilities of the plurality of users to each other.

### APPLICATION EXAMPLE 27

In the electronic apparatus according to the application example, the plurality of users may be users belonging to the same group.

With this configuration, it is easy to compare the users belonging to the same group. The same group is, for example, a group of users who all execute an exercise. For example, the same group is a mountaineering party moving together along the same route or a long-distance race team running along different sections of the same route in sequence.

### APPLICATION EXAMPLE 28

In the electronic apparatus according to the application example, a diagnosis result for each plurality of users may be output.

With this configuration, it is possible to evaluate the plurality of users objectively and fairly.

### APPLICATION EXAMPLE 29

In the electronic apparatus according to the application example, the diagnosis result may include at least one of a physical ability type of the user and advice for the user.

With this configuration, it is easy to plan content (route selection, role-sharing, and the like) of exercise of the plurality of users or achieve an improvement in safety or performance of all the plurality of users.

### APPLICATION EXAMPLE 30

In the electronic apparatus according to the application example, route information of a route along which the user exercises may be stored in association with the heart beat information.

The storage unit can manage the heart beat information in a state in which the heart beat information is associated with the route information. When the heart beat information which is an index calculation source is stored in the storage unit, the heart beat information can also be used for other usages as necessary.

### APPLICATION EXAMPLE 31

In the physical ability evaluation system according to the application example, the storage unit may store a comment in association with the associated route information and heart beat information.

The comment includes, for example, a comment such as an impression (hard, easy, or the like) of the user when the user thinks back on the exercise (mountaineering, running, or the like) related to the route or a memory (pleasant, boring, or the like). The physical ability evaluation system associates the comment with the route information and the heart beat information, it is possible to know relevance between the impression or the memory of the user and the actual heart beat information.

### APPLICATION EXAMPLE 32

In the electronic apparatus according to the application example, a comment may be stored in association with the associated route information and heart beat information.

The comment includes, for example, a comment such as an impression (hard, easy, or the like) of the user when the user thinks back on the exercise (mountaineering, running, or the like) related to the route or a memory (pleasant, boring, or the like). The electronic apparatus associates the comment with the route information and the heart beat information, it is possible to know relevance between the impression or the memory of the user and the actual heart beat information.

### BRIEF DESCRIPTION OF THE DRAWINGS

The invention will be described with reference to the accompanying drawings, wherein like numbers reference like elements.

FIG. 1 is an exemplary diagram illustrating a configuration of a system according to an embodiment.

FIG. 2 is an exemplary functional block diagram illustrating an electronic apparatus.

FIG. 3 is an exemplary functional block illustrating an information terminal, a main server, and a weather server.

FIG. 4 is a diagram illustrating an example of a worn sensor device.

FIG. 5 is an exemplary graph illustrating a relation between an elapsed time and a heart rate for each user of the same mountaineering party.

FIG. 6 is an exemplary graph illustrating a relation between a movement speed in the horizontal direction and a heart rate of a certain user.

FIG. 7 is a table illustrating a gear index, a slope ground adaptability index, and a stamina index for each user.

FIG. 8 is a diagram illustrating the gear index for each user.

FIG. 9 is a diagram illustrating the slope ground adaptability index for each user.

FIG. 10 is a diagram illustrating the stamina index for each user.

FIG. 11 is an exemplary flowchart illustrating extraction of the indexes from measurement data of a certain user moving along a mountaineering route.

FIG. 12 is an exemplary flowchart illustrating extraction of the indexes from measurement data of four users belonging to the same mountaineering party.

FIG. 13 is a diagram illustrating an exemplary radar chart to compare balances of various physical abilities among users.

FIG. 14 is a diagram illustrating an exemplary circular graph that shows a cumulative time of each class.

FIG. 15 is an exemplary flowchart illustrating a process of calculating a gradient based on positional information (a process performed when the positional information is acquired).

FIG. 16 is an exemplary flowchart illustrating a process of calculating a gradient based on weather information (a process performed when the positional information is acquired).

FIG. 17 is an exemplary flowchart illustrating a process of calculating a gradient based on an atmospheric difference between two points (a process performed when the positional information is acquired).

FIG. 18 is a diagram illustrating an exemplary format of measurement data.

FIG. 19 is a diagram illustrating a graph to compare slope ground adaptability of a plurality of users moving along the same route.

FIG. 20 is a diagram illustrating an example of measurement data acquired from a first user (a first electronic apparatus) along a certain route.

FIG. 21 is a diagram illustrating an example of measurement data acquired from a second user (a second electronic apparatus) along the same route.

FIG. 22 is an exemplary flowchart illustrating a process of managing a common slope degree based on the measurement data acquired from two different users (the first and second electronic apparatuses).

FIG. 23 is an exemplary table illustrating data of the slope degrees acquired through the process of FIG. 22.

FIG. 24 is a diagram illustrating an example of measurement data acquired from the first user (the first electronic apparatus) along a route.

FIG. 25 is a diagram illustrating an example of measurement data acquired from the second user (the second electronic apparatus) belonging to the same party along the same route.

FIG. 26 is a diagram illustrating an exemplary graph used when measurement data is clustered by atmospheric pressures.

FIG. 27 is a diagram illustrating an exemplary radar chart to compare balances of atmospheric pressures at which physical abilities can be exerted among users.

FIG. 28 is a diagram illustrating an exemplary radar chart that has five axes.

FIG. 29 is a diagram illustrating an exemplary radar chart to compare indexes for each atmospheric pressure.

FIG. 30 is a diagram illustrating an exemplary radar chart to compare indexes for each gradient.

FIG. 31 is a diagram illustrating an exemplary radar chart that has four axes.

FIG. 32 is a diagram illustrating an example of a screen on which diagnosis results are displayed.

FIG. 33 is a diagram illustrating an example of a screen on which advice is displayed.

### DESCRIPTION OF EXEMPLARY EMBODIMENTS

Hereinafter, preferred embodiments of the invention will be described in detail with reference to the drawings. The embodiments to be described below do not inappropriately limit content of the invention described in the appended claims. All of the configurations to be described below are not prerequisite configurations of the invention.

### 1. Server System

### 1-1. Configuration of Server System

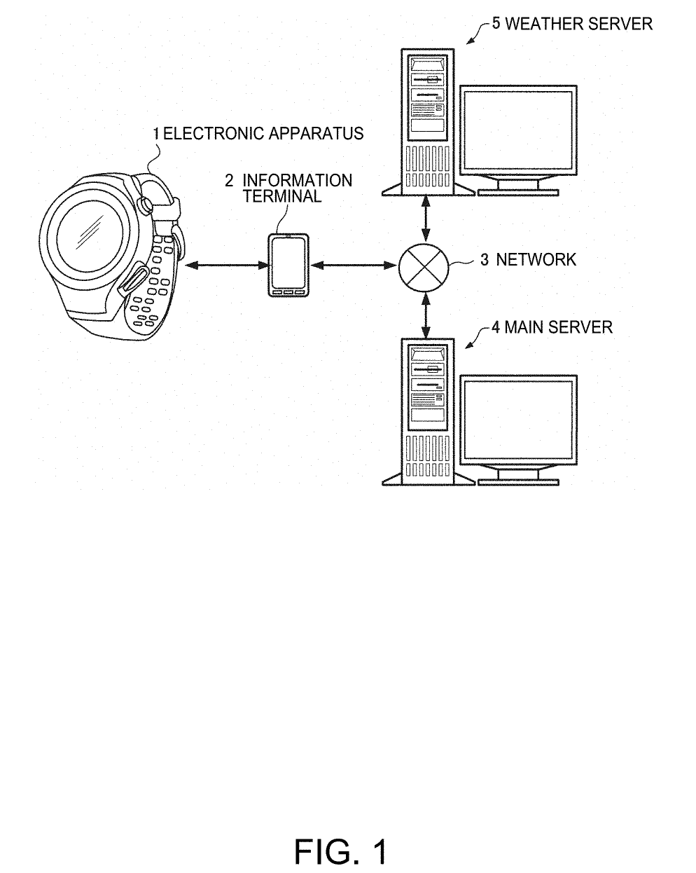

FIG. 1 is an exemplary diagram illustrating a configuration of a system according to an embodiment. As illustrated in FIG. 1, the system includes an electronic apparatus 1, an information terminal 2, a main server 4 (an example of a physical ability evaluation server), and a weather server 5. The information terminal 2, the main server 4, and the weather server 5 can be connected to a network 3 such as the Internet. Thus, the information terminal 2, the main server 4, and the weather server 5 can communicate with each other via the network 3. The electronic apparatus 1 can communicate with the information terminal 2 through short-range wireless communication or the like. Although not illustrated, the system according to the embodiment may include the plurality of electronic apparatuses 1 which are individually used by a plurality of users. The system according to the embodiment may include the plurality of information terminals 2 which are individually used by the plurality of users. One information terminal 2 may be shared by two or more users.

The electronic apparatus 1 is, for example, a portable information apparatus worn on a part of the body of a user in a sports scene such as mountaineering, trekking, or training. Here, “sports” mentioned in the present specification includes all of the movements of a user and may include movement of a route setting a summit as a goal, movement of a route unestablished setting a summit as a goal, movement (hiking) of rock climbing, snowy mountain climbing, trekking, or a flat route, movement of ups and downs or a flat route, movement by jogging, walking, or bicycling of ups and downs or a flat route, and training. Hereinafter, mountaineering is assumed as an example of sports. A wearing destination of the electronic apparatus 1 is, for example, a part (forearm) from an elbow to a hand of a user so that the electronic apparatus 1 can acquire measurement data related to an organism of the user in a contact or contactless manner or the user can view the electronic apparatus 1 as necessary. In the example illustrated in FIG. 1, the electronic apparatus 1 is configured as a wrist type (wristwatch type) electronic apparatus (outdoor watch) and a wearing destination of the electronic apparatus 1 is a wrist.

The information terminal 2 is an information terminal used by the user of the electronic apparatus 1 and is configured with, for example, a smartphone, a portable or desktop personal computer (PC), or a tablet PC.

The main server 4 is a server that supplies information regarding use of the electronic apparatus 1 to the user of the electronic apparatus 1 or manages measurement data acquired by the electronic apparatus 1 for user.

The weather server 5 is a server that supplies information regarding weather of each place to the main server 4 or the user using the electronic apparatus 1. In the embodiment, however, the information regarding weather may be directly supplied from the weather server 5 to the information terminal 2 or may be indirectly supplied to the information terminal 2 via the main server 4.

### 1-2. Configuration of Electronic Apparatus

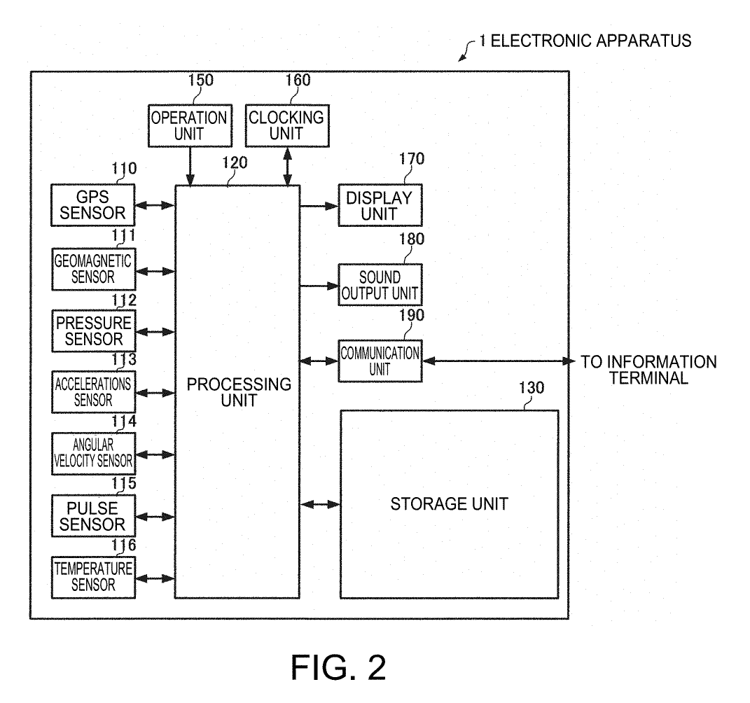

FIG. 2 is an exemplary functional block diagram illustrating the electronic apparatus 1.

As illustrated in FIG. 2, the electronic apparatus 1 is configured to include a GPS sensor 110, a geomagnetic sensor 111, a pressure sensor 112, an acceleration sensor 113, an angular velocity sensor 114, a pulse sensor 115, a temperature sensor 116, a processing unit 120, a storage unit 130, an operation unit 150, a clocking unit 160, a display unit 170, a sound output unit 180, and a communication unit 190. Here, in the configuration of the electronic apparatus 1, some of the constituent elements may be deleted or changed or other constituent elements (for example, a humidity sensor and an ultraviolet sensor) may be added.

The GPS sensor 110 is a sensor that generates positioning data (data of a latitude, a longitude, an altitude, a velocity vector, and the like) indicating a position or the like of the electronic apparatus 1 and outputs the positioning data to the processing unit 120 and is configured to include a Global Positioning System (GPS) receiver. The GPS sensor 110 receives electromagnetic waves including a satellite signal with a predetermined frequency bandwidth arriving from the outside through a GPS antenna (not illustrated), extracts a GPS signal from a GPS satellite, and generates positioning data indicating the position or the like of the electronic apparatus 1 based on the GPS signal.

The geomagnetic sensor 111 is a sensor that detects a magnetic field vector indicating a direction of the magnetic field of the earth viewed from the electronic apparatus 1 and generates, for example, geomagnetic data indicating a magnetic flux density in three axial directions perpendicular to each other. As the geomagnetic sensor 111, for example, a magnet resistive (MR) element, a magnet impedance (MI) element, or a Hall element is used.

The pressure sensor 112 is a sensor that detects a surrounding pressure (an atmospheric pressure) and includes, for example, a pressure-sensitive element of a scheme (vibration scheme) of using a change in a resonance frequency of a resonator element. The pressure-sensitive element is, for example, a piezoelectric vibrator formed of a piezoelectric material such as quartz crystal, lithium niobate, or lithium tantalate. For example, a tuning fork type vibrator, a dual tuning fork type vibrator, or an AT vibrator (thickness shear vibrator), or a SAW resonator is applied. Alternatively, the pressure sensor 112 may be, for example, a MEMS type pressure sensor manufactured using a semiconductor manufacturing technology. Specifically, the pressure sensor 112 includes a diaphragm unit that is flexural-deformed by a hydraulic pressure and a strain detection element that detects flexural deformation of the diaphragm unit. The diaphragm unit is formed of, for example, silicon. The strain detection element is, for example, a piezoresistive element. An output of the pressure sensor 112 may be used to correct the positioning data.

The acceleration sensor 113 is an inertial sensor that detects acceleration in each of triaxial directions intersecting each other (ideally, perpendicular to each other) and outputs a digital signal (acceleration data) according to the magnitude and direction of the detected triaxial acceleration. An output of the acceleration sensor 113 may be used to correct information regarding a position included in the positioning data of the GPS sensor 110.

The angular velocity sensor 114 is an inertial sensor that detects an angular velocity in each of triaxial directions intersecting each other (ideally, perpendicular to each other) and outputs a digital signal (angular velocity data) according to the magnitude and direction of the measured triaxial angular velocity. An output of the angular velocity sensor 114 may be used to correct information regarding a position included in the positioning data of the GPS sensor 110.

The pulse sensor 115 is a sensor that generates a signal indicating pulses (a heart rate) of the user and outputs the signal to the processing unit 120 and includes, for example, a light source such as an LED light source that emits measurement light with an appropriate wavelength to a hypodermic blood vessel and a light-receiving element that detects a change in the intensity of light generated from the blood vessel according to the measurement light. A function of calculating a heart rate per unit time (simply referred to as a “heart rate”) is mounted on the pulse sensor 115. The function of calculating a heart rate per unit time (an example of heart beat information) may be mounted on the processing unit 120. The pulse beat is obtained by indirectly measuring heart beats from a part (a wrist or the like) other than a heart. Since a pulse beat is highly correlated to a heart beat, the “pulse beat” and the “heart beat” are used as the same meaning in the present specification. An electrocardiographic sensor (electrocardiograph) that detects a weak potential of a cardiac muscle can also be employed in the pulse sensor 115.

The temperature sensor 116 is a thermosensitive element that outputs a signal according to surrounding temperature (for example, a voltage in accordance with temperature). The temperature sensor 116 may be a sensor that outputs a digital signal in accordance with temperature. The temperature sensor 116 includes a pressure-sensitive element of a scheme (vibration scheme) of using a change in a resonance frequency of a resonator element in accordance with surrounding temperature. The pressure-sensitive element is, for example, a piezoelectric vibrator formed of a piezoelectric material such as quartz crystal, lithium niobate, or lithium tantalate. For example, a tuning fork type vibrator, a dual tuning fork type vibrator, or an AT vibrator (thickness shear vibrator), or a SAW resonator is applied. Alternatively, the temperature sensor 116 may be a thermosensitive element that detects temperature by a thermocouple or a thermistor.

The storage unit 130 is configured with, for example, one IC memory or a plurality of IC memories and include a ROM that stores data such as a program and a RAM that serves as a working area of the processing unit 120. The RAM also includes a nonvolatile RAM.

The operation unit 150 is configured to have, for example, a button, a key, a microphone, a touch panel, a sound recognition function (using a microphone (not illustrated)), and an action detection function (using the acceleration sensor 113 or the like) and performs processes of converting an instruction from the user into an appropriate signal and transmitting the signal to the processing unit 120.

The clocking unit 160 is configured with, for example, a real time clock (RTC) IC, generates time data such as year, month, day, hour, minute, and second, and transmits the time data to the processing unit 120.

The display unit 170 is configured with, for example, a liquid crystal display (LCD), an organic electroluminescence (EL) display, an electrophoretic display (EPD), or a touch panel display and displays various images in response to an instruction from the processing unit 120.

The sound output unit 180 is configured with, for example, a speaker, a buzzer, or a vibrator and generates various sounds (or vibration) in response to an instruction from the processing unit 120.

The communication unit 190 performs various kinds of control to establish data communication between the electronic apparatus 1 and the information terminal 2 (a smartphone or the like). The communication unit 190 is configured with, for example, a transceiver corresponding to a short-range wireless communication standard such as Bluetooth (registered trademark) (including Bluetooth Low Energy (BTLE)), wireless fidelity (Wi-Fi) (registered trademark), Zigbee (registered trademark), near field communication (NFC), or ANT+(registered trademark).

The processing unit 120 is configured with, for example, a microprocessing unit (MPU), or a digital signal processor (DSP), an application specific integrated circuit (ASIC). The processing unit 120 performs various processes in accordance with programs stored in the storage unit 130 and various commands input via the operation unit 150 by the user. The processes performed by the processing unit 120 include data processing on signals output by the GPS sensor 110, the geomagnetic sensor 111, the pressure sensor 112, the acceleration sensor 113, the angular velocity sensor 114, the pulse sensor 115, the temperature sensor 116, and the clocking unit 160, a display process of causing the display unit 170 to display an image, and a sound output process of causing the sound output unit 180 to output a sound. The processing unit 120 performs processes of receiving a control command from the information terminal 2 via the communication unit 190 or various calculation processes on data received from the information terminal 2 via the communication unit 190 in accordance with various programs. The processing unit 120 performs processes of reading data from the storage unit 130 and transmitting the data with a predetermined format to the information terminal 2 via the communication unit 190 in accordance with various programs. The processing unit 120 performs processes of transmitting various kinds of information to the information terminal 2 via the communication unit 190 and displaying various screens based on information received from the information terminal 2 in accordance with various programs. The processing unit 120 performs other various control processes. For example, the processing unit 120 performs a process of causing the display unit 170 to display an image (an image, a moving image, text, a sign, or the like) based on information received by the communication unit 190 or at least a part of the information stored in the storage unit 130. The electronic apparatus 1 may include a vibration mechanism. The vibration mechanism may convert various kinds of information into vibration information and notifies the user of the vibration information.

### 1-3. Configuration of Information Terminal

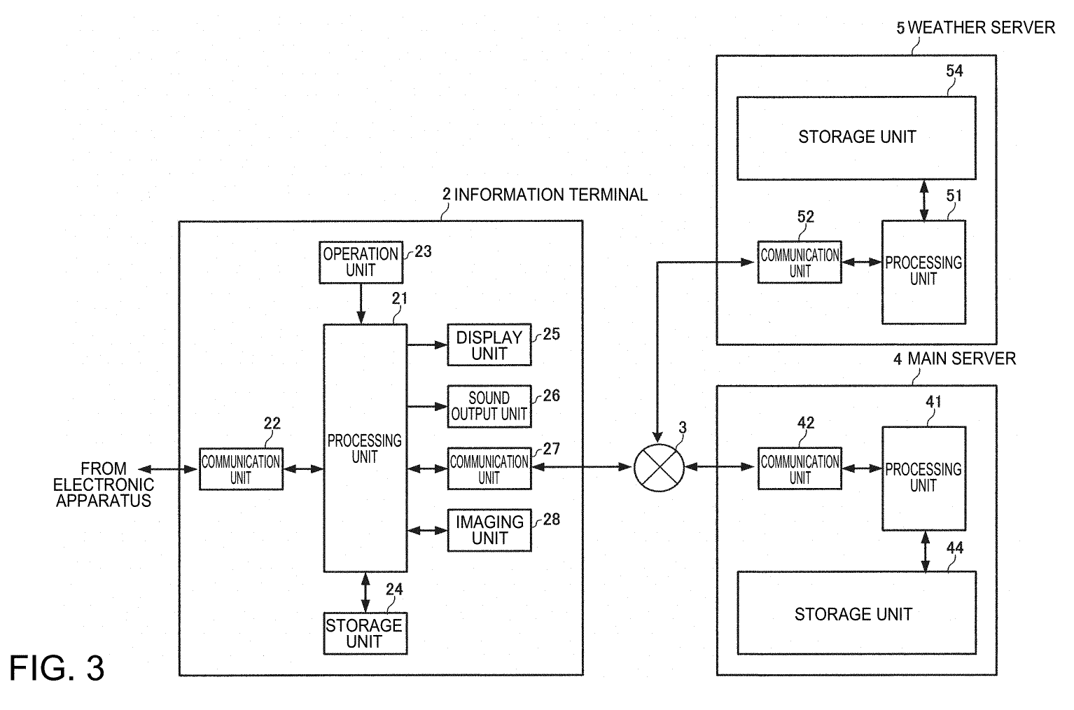

The drawing illustrated on the left side on the sheet surface of FIG. 3 is an exemplary functional block diagram illustrating the information terminal 2. As illustrated in FIG. 3, the information terminal 2 is configured to include a processing unit 21, a communication unit 22, an operation unit 23, a storage unit 24, a display unit 25, a sound output unit 26, a communication unit 27, and an imaging unit 28. Here, in the configuration of the information terminal 2, some of the constituent elements may be appropriately deleted or changed or other constituent elements may be added.

The communication unit 22 performs a process of receiving data (measurement data) or the like transmitted with a predetermined format from the electronic apparatus 1 and transmitting the data to the processing unit 21 and a process of transmitting a control command from the processing unit 21 to the electronic apparatus 1.

The operation unit 23 performs a process of acquiring data in accordance with an operation by the user and transmitting the data to the processing unit 21. The operation unit 23 may be, for example, a touch panel type display, a button, a key, or a microphone.

The storage unit 24 is configured with, for example, any of various integrated circuit (IC) memories such as a read-only memory (ROM), a flash ROM, and a random access memory (RAM) or a recording medium such as a hard disk or a memory card. The storage unit 24 stores a program causing the processing unit 21 to perform various calculation processes or control processes or data or various programs for realizing application functions. The storage unit 24 is used as a working area of the processing unit 21 and temporarily stores data acquired by the operation unit 23 and arithmetic results or the like executed by the processing unit 21 in accordance with various programs. Further, the storage unit 24 may store data necessarily stored for a long time among the data generated through the process of the processing unit 21.

The display unit 25 displays a processed result of the processing unit 21 as text, a graph, an animation, and another image. The display unit 25 may be, for example, a cathode ray tube (CRT), a liquid crystal display (LCD), an electrophoretic display (EPD), a touch panel type display, or a head-mounted display (HMD). The functions of the operation unit 23 and the display unit 25 may be realized as one touch panel type display.

The sound output unit 26 outputs a processed result of the processing unit 21 as a voice or a sound such as a buzzer sound. The sound output unit 26 may be, for example, a speaker or a buzzer.

The communication unit 27 performs data communication with the communication unit 42 of the main server 4 via the network 3. For example, the communication unit 27 performs a process of receiving data from the processing unit 21 and transmitting the data with a predetermined format to the communication unit 42 of the main server 4. For example, the communication unit 27 performs a process of receiving information necessary to display a screen from the communication unit of the main server 4 and transmitting the information to the processing unit 21 and a process of receiving various kinds of information from the processing unit 21 and transmitting the various kinds of information to the communication unit of the main server 4.

The imaging unit 28 is a camera that includes a lens, a color image sensor, and a focus adjustment mechanism. An image of a subject in a field formed by the lens is formed by the image sensor. Data (image data) of the image acquired by the image sensor is transmitted to the processing unit 21 and is stored in the storage unit 24 or displayed on the display unit 25.

The processing unit 21 is configured with a central processing unit (CPU), a digital signal processor (DSP), an application specific integrated circuit (ASIC), or the like. The processing unit 21 performs various processes in accordance with programs stored in the storage unit 24 and various commands input by the user via the operation unit 23. The processes by the processing unit 21 includes data processing on data generated by the information terminal 2, a display process of displaying an image on the display unit 25, a sound output process of outputting a sound to the sound output unit 26, and image processing on an image acquired by the imaging unit 28. The processing unit 21 may be configured with a single processor or may be configured with a plurality of processors. The processing unit 21 performs a process of transmitting a control command to the electronic apparatus 1 via the communication unit 22 or various calculation processes on data received from the electronic apparatus 1 via the communication unit 22 in accordance with various programs. The processing unit 21 performs a process of reading data from the storage unit 24 and transmitting the data with a predetermined format to the main server 4 via the communication unit 27 in accordance with any of various programs. The processing unit 21 performs a process of transmitting various kinds of information to the main server 4 via the communication unit 27 and displaying various screens based on the information received from the main server 4 in accordance with any of various programs. The processing unit 21 performs other various control processes. For example, the processing unit 21 performs a process of displaying an image (an image, a moving image, text, a sign, or the like) on the display unit 25 based on at least some of information received by the communication unit 27, information received by the communication unit 22, and the information stored in the storage unit 24. The information terminal 2 or the electronic apparatus 1 may include a vibration mechanism and the vibration mechanism may convert various kinds of information into vibration information and notify the user of the vibration information.

### 1-4. Configuration of Main Server

The drawing illustrated on the bottom right side on the sheet surface of FIG. 3 is an exemplary functional block diagram illustrating the main server 4. As illustrated on the right bottom side of the sheet surface of FIG. 3, the main server 4 is configured to include a processing unit 41 (an example of a computer), a communication unit 42, and a storage unit 44. Here, in the configuration of the main server 4, some of the constituent elements may be appropriately deleted or changed or other constituent elements may be added.

The storage unit 44 is configured with, for example, any of various IC memories such as a ROM, a flash ROM, and a RAM or a recording medium such as a hard disk or a memory card. The storage unit 44 stores a program (an example of a physical ability evaluation program) causing the processing unit 41 to perform various calculation processes or control processes or data or various programs for realizing an application function (an example of a physical ability evaluation method). The storage unit 44 is used as a working area of the processing unit 41 and temporarily stores arithmetic results or the like executed by the processing unit 41 in accordance with various programs. Further, the storage unit 44 may store data necessarily stored for a long time among the data generated through the process of the processing unit 41. Various kinds of information stored in the storage unit 44 will be described below.

The communication unit 42 performs data communication with the communication unit 27 of the information terminal 2 via the network 3. For example, the communication unit 42 performs a process of receiving data from the communication unit 27 of the information terminal 2 and transmitting the data to the processing unit 41. For example, the communication unit 42 performs a process of transmitting information necessary to display a screen with a predetermined format to the communication unit 27 of the information terminal 2 and a process of receiving information from the communication unit 27 of the information terminal 2 and transmitting the information to the processing unit 41.

The processing unit 41 performs a process of receiving data from the information terminal 2 via the communication unit 42 and storing the data in the storage unit 44 in accordance with any of various programs. The processing unit 41 performs a process of receiving various kinds of information from the information terminal 2 via the communication unit 42 and transmitting information necessary to display various screens to the information terminal 2 in accordance with any of various programs. The processing unit 41 performs other various control processes.

### 1-5. Configuration of Weather Server

The drawing illustrated on the top right side on the sheet surface of FIG. 3 is an exemplary functional block diagram illustrating the weather server 5. As illustrated on the right bottom side of the sheet surface of FIG. 3, the weather server 5 is configured to include a processing unit 51, a communication unit 52, and a storage unit 54. Here, in the configuration of the weather server 5, some of the constituent elements may be appropriately deleted or changed or other constituent elements may be added.

The storage unit 54 is configured with, for example, any of various IC memories such as a ROM, a flash ROM, and a RAM or a recording medium such as a hard disk or a memory card. The storage unit 54 stores a program causing the processing unit 51 to perform various calculation processes or control processes or data or various programs for realizing an application function. The storage unit 54 is used as a working area of the processing unit 51 and temporarily stores arithmetic results or the like executed by the processing unit 51 in accordance with various programs. Further, the storage unit 54 may store data necessarily stored for a long time among the data generated through the process of the processing unit 51. Various kinds of information stored in the storage unit 54 will be described below.

The communication unit 52 performs data communication with the communication unit 27 of the information terminal 2 via the network 3. For example, the communication unit 52 performs a process of receiving data from the communication unit 27 of the information terminal 2 and transmitting the data to the processing unit 51. For example, the communication unit 52 performs a process of transmitting information necessary to display a screen with a predetermined format to the communication unit 27 of the information terminal 2 and a process of receiving information from the communication unit 27 of the information terminal 2 and transmitting the information to the processing unit 51.

The processing unit 51 performs a process of receiving data from the information terminal 2 via the communication unit 52 and storing the data in the storage unit 54 in accordance with any of various programs. The processing unit 51 performs a process of receiving various kinds of information from the information terminal 2 via the communication unit 52 and transmitting information necessary to display various screens to the information terminal 2 in accordance with any of various programs. The processing unit 51 performs other various control processes.

### 1-6. Use of Sensor Device

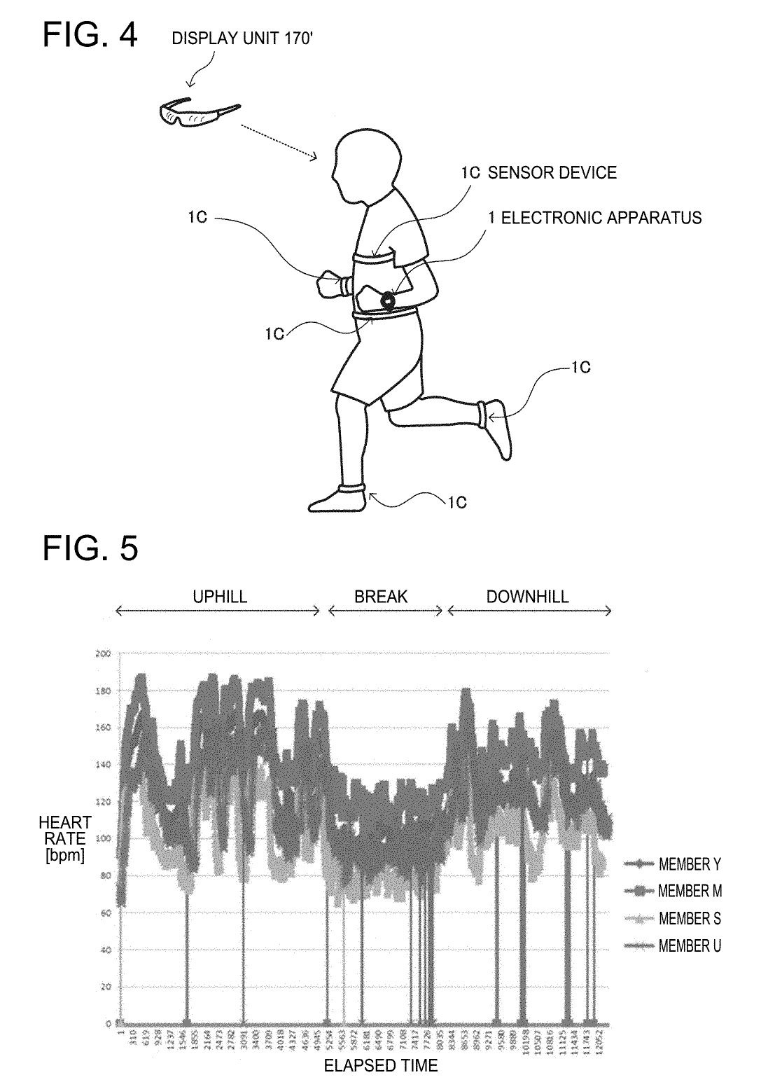

The user of the electronic apparatus 1 may use a sensor device 1C instead of the electronic apparatus 1 or along with the electronic apparatus 1. A wearing destination of the sensor device 1C can be appropriately selected by the user in accordance with a purpose of training or the like and is, for example, one of a head, an upper arm, a forearm, a waist, a chest, a thigh, a lower thigh, a foot of the user (see FIG. 4). When the sensor device 1C is worn on a part of the physical body, a wearing tool (a belt, a clip, or the like) appropriate for the spar of the part or the shape of a training wear or the like may be used. The sensor device 1C is configured to include a GPS sensor, a geomagnetic sensor, a pressure sensor, an acceleration sensor, an angular velocity sensor, a pulse sensor, a temperature sensor, a processing unit, a storage unit, an operation unit, a clocking unit, a display unit, a sound output unit, and a communication unit.

The GPS sensor, the geomagnetic sensor, the pressure sensor, the acceleration sensor, the angular velocity sensor, the pulse sensor, the temperature sensor, the processing unit, the storage unit, the operation unit, the clocking unit, the display unit, the sound output unit, and the communication unit mounted on the sensor device IC have the same functions as the GPS sensor, the geomagnetic sensor, the pressure sensor, the acceleration sensor, the angular velocity sensor, the pulse sensor, the temperature sensor, the processing unit, the storage unit, the operation unit, the clocking unit, the display unit, the sound output unit, and the communication unit mounted on the electronic apparatus 1.

The sensor device 1C can communicate with the electronic apparatus 1 via the communication unit of the sensor device 1C and the communication unit of the electronic apparatus 1. Measurement data acquired by the GPS sensor, the geomagnetic sensor, the pressure sensor, the acceleration sensor, and the angular velocity sensor, the pulse sensor, and the temperature sensor of the sensor device 1C is read at an appropriate timing by the electronic apparatus 1. The measurement data read by the electronic apparatus 1 is uploaded at an appropriate timing to the main server 4.

When the sensor device 1C is used instead of the electronic apparatus 1, the sensor device IC is preferably configured to communicate with the information terminal 2 via the communication unit of the sensor device IC and the communication unit of the information terminal 2.

A head-mounted display unit 170′ can be used as the display unit of the electronic apparatus 1 or the display unit of the information terminal 2. The display unit 170′ is a so-called head-mounted display that displays information in front of the eyes of the user.

### 1-7. Operation of User

### 1-7-1. Registration of Mountaineering Plan in Main Server

Next, an operation of the user will be described with reference to FIG. 1.

The user operates the information terminal 2 to access the main server 4 via the network 3 and generate a file (for example, a GPX file) of data of a mountaineering plan using information regarding a map supplied from the main server 4 via the network 3.

The file (for example, a file with a GPX format) of the data of the mountaineering plan includes (i) a title (plan name) of the mountaineering plan, (ii) positional coordinates of each point forming a mountaineering route, (iii) a mountain ascending date, (iv) a mountain descending date, (v) positional coordinates of a mountain ascending entrance, (vi) positional coordinates of a mountain descending exit, (vii) positional coordinates of route representative spot (a summit, a break location, or the like), (viii) a whole length of the mountaineering route, and (ix) an altitude difference of the mountaineering route. Of these items, information of (ii) is written in the file of the data of the mountaineering plan and information of (i) and (iii) to (ix) is written in tag information of the file of the data of the mountaineering plan. Measurement data measured by the electronic apparatus 1 in actual mountaineering is also appropriate written in the file of the data of the mountaineering plan. Hereinafter, the data of the mountaineering plan including the data of the mountaineering route is also referred to as “data of the mountaineering route.”

The user uploads the file (for example, a file with a GPX format) of the data of the generated mountaineering plan to the main server 4. At this time, the file (for example, a file with a GPX format) of the data of the mountaineering plan is registered in a database assigned by the user in the storage unit 54 of the main server 4. Accordingly, the user can store the files of the data of the plurality of mountaineering plans generated by the user in the main server 4. Of the files of the data of the mountaineering plans registered in the database, at least one file can also be opened to other users. The user can also store at least one of files of data of mountaineering plans opened by other users as at least one of a file of data of a mountaineering plan of the user in the main server 4.

Since processes of the information terminal 2 and the main server 4 at the time of registering the files of the data of the mountaineering plans in the main server 4 are known, the description thereof will be omitted here.

An application program necessary when the information terminal 2 and the main server 4 transmit and receive information is installed in advance in the storage unit 24 of the information terminal 2, the application program is assumed to be used for registration of the files of the data of the mountaineering plans. The application program may be written in the storage unit 24 from a storage medium (not illustrated).

### 1-7-2. Writing of Data of Mountaineering Plan in Electronic Apparatus

In a state in which the electronic apparatus 1 is connected to the information terminal 2, the user accesses the main server 4 from the information terminal 2 and writes the file of the data of the mountaineering plan added to the main server 4 in the electronic apparatus 1 through the information terminal 2. The file of the data of the mountaineering plan written in the electronic apparatus 1 is used for a navigation program or the like when the user actually executes mountaineering in accordance with the data of the mountaineering plan. Instead of writing the file of the data of the mountaineering plan in the electronic apparatus 1, only data of a mountaineering route included in the file of the data of the mountaineering plan may be written with a predetermined format in the electronic apparatus 1.

Since a process of the electronic apparatus 1 at the time of writing the file of the data of the mountaineering plan in the electronic apparatus 1 is known, the description thereof will be omitted here.

In the writing of the file of the data of the mountaineering plan in the electronic apparatus 1, the above-described application program is assumed to be used.

### 1-7-3. Upload of Measurement Data to Main Server

After the user executes the mountaineering using the data of the mountaineering plan (including the data of the mountaineering route), the user connects the electronic apparatus 1 to the information terminal 2 to upload the measurement data acquired by various sensors of the electronic apparatus 1 during the mountaineering to the main server 4 via the information terminal 2. The measurement data is written in the corresponding file (the file of the data of the corresponding mountaineering plan) in the database of the main server 4. Accordingly, the user can store the measurement data of the user during the mountaineering in the main server 4. The measurement data can be helpful in managing health of the user and improving performance.

Since processes of the information terminal 2 and the main server 4 at the time of writing the measurement data in the main server 4 are known, the description thereof will be omitted here.

The above-described application program is assumed to be used to store the measurement data in the main server 4.

### 1-8. Data Stored by System

### 1-8-1. Data Stored in Storage Unit of Electronic Apparatus

For example, the following data is stored in the storage unit 130 of the electronic apparatus 1.

### (1) Program

A program which is executed by the processing unit 120 of the electronic apparatus 1 is stored. The program includes a measurement program that drivers at least one of the sensors (the GPS sensor 110, the geomagnetic sensor 111, the pressure sensor 112, the acceleration sensor 113, the angular velocity sensor 114, the pulse sensor 115, and the temperature sensor 116) mounted on the electronic apparatus 1 and generates measurement data based on an output (sensing data) of the sensor. The program may include a navigation program that guides the user to the mountaineering plan based on the data of the mountaineering plan stored in the storage unit 130 of the electronic apparatus 1.

### (2) Measurement Data

The measurement data acquired by at least one of the sensor (the GPS sensor 110, the geomagnetic sensor 111, the pressure sensor 112, the acceleration sensor 113, the angular velocity sensor 114, the pulse sensor 115, and the temperature sensor 116) mounted on the electronic apparatus 1 is stored. The measurement data is, for example, data associating an output of the sensor each time with the time.

### (3) Data of Mountaineering Plan

The file of the data of the mountaineering plan written by the information terminal 2 is stored. The file of the data of the mountaineering plan includes data of at least one mountaineering route. The data of the mountaineering route is data of positional coordinates of each point forming the mountaineering route. The data of the data of the mountaineering plan also includes various kinds of information subordinate to the mountaineering route such as a plan name, a mountain ascending date, a mountain descending data, positional coordinates of the mountain ascending entrance, positional coordinates of the mountain descending exit, positional coordinates of a route representative spot (a summit, a break location, or the like), a whole length of the mountaineering route, and an altitude difference of the mountaineering route.

### 1-8-2. Data Stored in Storage Unit of Information Terminal

For example, the following data is stored in the storage unit 24 of the information terminal 2.

### (1) Program

A program which is executed by the processing unit 21 of the information terminal 2 is stored. The program is a program (application program) for receiving supply of necessary information from the main server 4.

### (2) Temporary Stored Data

The measurement data read from the electronic apparatus 1, data downloaded from the main server 4, and the like are stored. A period in which the data is stored in the storage unit 24 of the information terminal 2 may be temporary.

### 1-8-3. Data Stored in Storage Unit of Main Server

For example, the following data is stored in the storage unit 44 of the main server 4 is stored.

### (1) Program

A program which is executed by the processing unit 41 of the main server 4 is stored.

### (2) Database for Each User

A database that accumulates the file of the data of the mountaineering plan stored by the user via the information terminal 2 is stored. For example, the file of the data of the mountaineering plan is managed for each user and each date. Here, the file of the data of the mountaineering plan includes a file of the data of the mountaineering plan generated by the user using a main application program. The measurement data uploaded the user through the information terminal 2 is written in this file. The measurement data of mountaineering executed in accordance with the mountaineering plan is written in the file of the data of the mountaineering plan.

### 1-8-4. Data Stored in Storage Unit of Weather Server

For example, the following data is stored in the storage unit 54 of the weather server 5.

### (1) Program

A program which is executed by the processing unit 51 of the weather server 5 is stored.

### (2) Weather Data

Data related to weather such as temperature, humidity, atmospheric pressure, and a rainfall amount is stored. The data related to the weather includes actually measured data and predicted data.

### 1-9. Embodiment

### 1-9-1. Overview of Embodiment

The system (an example of a physical ability evaluation system) according to the embodiment includes an output unit that outputs a plurality of indexes (a stamina index, a gear index, a slope ground adaptability index, and the like to be described below) related to heart beat information of a user as information related to balance of the physical ability of the user. In the related art, the user merely experimentally grasped to which type the user is relevant among various types such as a type strong in a long distance, a type strong to a short distance, a type strong an uphill ground, a type strong to a downhill ground, and a type strong to a flat ground. However, according to the information (that is, the information related to the balance) which is based on the plurality of indexes related to the heart beat information, the user can objectively grasp his or her physical ability type. The “heart beat information” includes information related to the number of cardiac beats per unit time, such as information regarding a heart rate, information regarding a pulse rate, and electrocardiographic information. The output unit can be configured with a processing unit, a display unit, a sound output unit, a communication unit, or the like. An output destination of the index may be a user or a device. Hereinafter, a case in which the processing unit 41 or the communication unit 42 of the main server 4 or the communication unit 22, the processing unit 21, or the display unit 25 of the information terminal 2 are used as an output unit will be described.

In the system according to the embodiment, the plurality of indexes include at least one of an index (a stamina index to be described below) indicating the degree of a change in the heart beat information with respect to an elapsed time of the user, an index (a gear index to be described below) indicating the degree of a change in the heart beat information with respect to a change in a speed of the user in a horizontal direction perpendicular to a gravity direction, and an index (a slope ground adaptability index to be described below) indicating the degree of a change in the heart beat information with respect to a speed of the user the gravity direction.

An energy consumption rate of the user during exercise or stop is reflected in the index which is based on the elapsed time (a stamina index to be described below) among the plurality of indexes. Therefore, in particular, the energy consumption rate among individual user physical abilities can be evaluated objectively and simply with the index (the energy consumption rate mentioned here is assumed to include meaning of an energy recovery rate and is also referred to as “stamina”).

Conversion efficiency from beating of the heart of the user who is exercising to a movement speed in the horizontal direction is reflected in the index (corresponding to a gear index to be described below) which is based on the change in the speed in the horizontal direction among the plurality of indexes. Therefore, in particular, horizontal movement efficiency can be evaluated objectively and simply with the index among the individual user physical abilities.

Conversion efficiency from beating of the heart of the user who is exercising to a movement speed in the gravity direction is reflected in the index (a slope ground adaptability index to be described below) which is a based on the change in the speed in the gravity direction among the plurality of indexes. Therefore, in particular, movement efficiency in the gravity direction can be evaluated objectively and simply among the individual user physical abilities.

Accordingly, in the system according to the embodiment, the balance of the plurality of indexes (the stamina index, the gear index, the slope ground adaptability index) can be output as information related to the balance of the physical ability of the user.

In the system according to the embodiment, the electronic apparatus 1 records the measurement data including biological information and exercise information of the user when the user is moving amount the mountaineering route, in the storage unit 130. The measurement data is uploaded to the storage unit 44 of the main server 4 via the information terminal 2. The processing unit 41 of the main server 4 evaluates the physical ability of the user at various angles by appropriately classifying (clustering) and analyzing the measurement data.

Here, the biological information in the measurement data includes, for example, data of a heart rate and data of body temperature. The heart rate can be calculated based on, for example, an output of the pulse sensor 115 and the body temperature can be calculated based on, an output of the temperature sensor 116.

The exercise information in the measurement data includes data of a movement speed of the user and data of a slope degree at each spot of the mountaineering route along which the user has moved. Incidentally, a movement speed in the gravity direction (the vertical direction), a movement speed in the horizontal direction (a direction perpendicular to the gravity direction), a slope degree of the mountaineering route can be calculated based on, for example, an output of the GPS sensor 110 and an output of the pressure sensor 112 (the details will be described below).

According to the embodiment, the user wears the electronic apparatus 1, moves along the mountaineering route, accumulates the measurement data during the movement in the storage unit 130 of the electronic apparatus 1. After the mountaineering, the user connects the electronic apparatus 1 to the information terminal 2 at his or her home to upload the measurement data of the storage unit 130 to the main server 4 via the information terminal 2 and the network 3. The processing unit 41 of the main server 4 registers the measurement data in the database of the user. At this time, the uploaded measurement data is written in the file of the data of the corresponding mountaineering plan in the database. Thus, the measurement data is registered in the database. The measurement data may be uploaded (registered in the database) in sequence during the mountaineering.

A storing destination of the database may be the storage unit 130 of the electronic apparatus 1 in addition to the storage unit 44 of the main server 4 or may be the storage unit 24 of the information terminal 2. Hereinafter, the storing destination of the database is assumed to be the storage unit 44 of the main server 4.

### 1-9-2. Data of Mountaineering Route

In the system according to the embodiment, the storage unit 44 of the main server 4 stores the heart beat information in association with the data of the mountaineering route. In this case, the storage unit 44 can manage the heart beat information in the state in which the heart beat information is associated with the data of the mountaineering route. When the heart beat information which is an index calculation source is stored in the storage unit 44, the heart beat information can also be used for other usages as necessary. Hereinafter, specific description will be made.

In the system according to the embodiment, the heart beat information can be associated with the data of the mountaineering route (an example of the route information) along which the user exercises. In this case, the route along which the user exercises can indirectly match the index related to the physical ability of the user. By this matching, it is possible to analyze a change in the physical ability which depends on the route. For example, it is possible to distinguish a route (or a section) along which the user exerts his or her physical ability from a route (or a section) along which the user may not exert his or her physical ability.

In the system according to the embodiment, the data of the mountaineering route includes at least one of positional information in the horizontal direction perpendicular to the gravity direction and positional information in the gravity direction. In this case, it is possible to analyze a change in the physical ability which depends on at least one of an altitude and a spot at which the user exercises. For example, it is possible to distinguish the altitude (or an area) at which the user can exercise his or her physical ability from an altitude (or an area) along which the user may not exert his or her physical ability.

Specifically, in the system according to the embodiment, the data of the mountaineering route is generated as follows.

As described above, measurement data acquired in actual mountaineering is written in the data of the mountaineering plan registered in the database. The measurement data includes at least data of a heart rate of the user at each time and data of the position (including an altitude) of the user at each time. The data of the heart rate is based on an output of the pulse sensor 115 and the data of the position is based on an output of the GPS sensor 110.

Accordingly, the processing unit 41 of the main server 4 generates the data of the mountaineering route along which the user has actually moved by arranging the data at positions included in the measurement data in a chronological order. The generated data of the mountaineering route is written in the file of the data of the mountaineering plan.

The processing unit 41 of the main server 4 calculates a gradient (slope degree) of each section (each spot) based on data of the position of each section (each spot) forming the mountaineering route and determines whether each section (each spot) belongs to an uphill ground, a downhill ground, or a flat ground. A determination result (classification of each section (each spot)) of each section (each spot) is written in the file of the data of the mountaineering plan in a state in which the determination result is associated with the measurement data of the section (the spot).

The processing unit 41 of the main server 4 can detect a spot at which the position of the user is not changed for a given period or more based on a temporal change in the position of the user in the mountaineering route as a break spot of the user in the mountaineering route. Information indicating whether each section (each spot) is the break spot is assigned to the measurement data of the section (the spot).

Here, the case in which the data of the mountaineering route is generated from the measurement data acquired in the actual mountaineering has been described, but the data of the mountaineering route stored in advance in the file of the data of the mountaineering plan can also be used as the data of the mountaineering route without change.

The databases of the plurality of users are assumed to be individually stored in the storage unit 54 of the main server 4. The data of the users executing the mountaineering together based on the same mountaineering plan is assumed to be associated with each other.

For example, in a case in which four users, members Y, M, S, and U has executed the same mountaineering plan for the same period, members M, S, and U can be associated as fellow travelers with the data of the mountaineering plan of member Y, members Y, S, and U can be associated as fellow travelers with the data of the mountaineering plan of member M, members Y, M, and U can be associated as fellow travelers with the data of the mountaineering plan of member S, and members Y, M, and S can be associated as fellow travelers with the data of the mountaineering plan of member U. The associated information may be uploaded along with the data of the mountaineering plan to the main server 4 by, for example, a representative (a person generating the data of the mountaineering plan) among the four users.

### 1-9-3. Overview of Indexes

In the embodiment, the indexes indicating the physical ability of the user are calculated based on the database of the user. An overview of the indexes is as follows.

### (1) Stamina Index

A stamina index is an index for evaluating the degree of a change in a heart rate with reference to an “elapsed time” when the user is moving or stops the mountaineering route. Stamina (an energy consumption rate or recovery rate) of the user is reflected in this index. The energy consumption rate is reflected in the index during the movement of the user. The energy recovery rate is reflected in the index during the stop of the user. The processing unit 41 according to the embodiment calculates the index separately for an uphill ground, a downhill ground, a flat ground, and stop. That is, in the system according to the embodiment, the heart beat information used to calculate the stamina index may be heart beat information detected when the user is exercising. In this case, the energy consumption rate during the exercise of the user can be output as the index. In the system according to the embodiment, the heart beat information used to calculate the stamina index may be heart beat information detected when the user stops. In this case, the energy recovery rate during the stop of the user can be output as the index.

### (2) Efficiency Index (Gear Index)

An efficiency index is an index for evaluating the degree of a change in a heart rate with reference to a “movement speed in the horizontal direction” when the user is moving along the mountaineering route. Efficiency at which the user converts beating of his or her heart into movement energy in the horizontal direction is reflected in the index. The processing unit 41 according to the embodiment calculates the index separately for an uphill ground, a downhill ground, and a flat ground.

### (3) Slope Ground Adaptability Index

A slope ground adaptability index is an index for evaluating the degree of a change in a heart rate with reference to a “movement speed in the gravity direction” when the user is moving along the mountaineering route. Efficiency at which the beating of the user converts beating into energy for hill ascending or descending is reflected in the index. The processing unit 41 according to the embodiment calculates the index separately for an uphill ground and a downhill ground.

### 1-9-4. Method of Calculating Stamina Index

A method of calculating the stamina index is as follows.

(1) The processing unit 41 of the main server 4 registers heart rates or the like of the user moving various mountaineering routes at each time in the database.

(2) The processing unit 41 of the main server 4 classifies (clusters) sections (or spots) of a statistical target route into an uphill ground, a flat ground, a downhill ground, and a break spot based on the gradients (slope degrees) of the sections (or the spots). That is, the processing unit 41 clusters the measurement data of the spots in accordance with the gradients (the slope degrees) (in addition, in the clustering, information regarding a movement speed of the user may be used to detect a break spot). The statistical target route may be a partial route of a specific mountaineering route, may be a specific mountaineering route, may be a specific mountaineering route used for a specific period, or may be a whole mountaineering route.

(3) The processing unit 41 of the main server 4 plots measurement data of each uphill ground section to an elapsed time-heart rate graph, obtains an elapsed time-heart rate curve (straight line) by regression analysis, and obtains an inclination of the curve as an “uphill ground stamina index”.

(4) The processing unit 41 of the main server 4 plots measurement data of each downhill ground section to an elapsed time-heart rate graph, obtains an elapsed time-heart rate curve (straight line) by regression analysis, and obtains an inclination of the curve as a “downhill ground stamina index”.

(5) The processing unit 41 of the main server 4 plots measurement data of each flat ground section to an elapsed time-heart rate graph, obtains an elapsed time-heart rate curve (straight line) by regression analysis, and obtains an inclination of the curve as a “flat ground stamina index”.

(6) The processing unit 41 of the main server 4 obtains an elapsed time-heart rate curve (straight line) at each break spot by regression analysis and obtains an inclination of the curve as a “break stamina index”.

(7) The mountaineering route is classified into the uphill ground, the flat ground, the downhill ground, and the break spot here, but may not be classified. A stamina index obtained without classification is simply referred to as a “stamina index”.

### 1-9-5. Method of Calculating Efficiency Index (Gear Index)

A method of calculating the efficiency index (gear index) is as follows.

(1) The processing unit 41 of the main server 4 registers heart rates or the like of the user moving various mountaineering routes at each time in the database.

(2) The processing unit 41 of the main server 4 classifies (clusters) sections (or spots) of a statistical target route into an uphill ground, a flat ground, and a downhill ground based on the gradients (slope degrees) of the sections (or the spots). That is, the processing unit 41 clusters the measurement data of the spots in accordance with the gradients (the slope degrees). The statistical target route may be a partial route of a specific mountaineering route, may be a specific mountaineering route, may be a specific mountaineering route used for a specific period, or may be a whole mountaineering route.

(3) The processing unit 41 of the main server 4 plots measurement data of each uphill ground section to a horizontal direction speed-heart rate graph, obtains a horizontal direction speed-heart rate curve (straight line) by regression analysis, and obtains an inclination of the curve as an “uphill ground gear index”.

(4) The processing unit 41 of the main server 4 plots measurement data of each downhill ground section to a horizontal direction speed-heart rate graph, obtains a horizontal direction speed-heart rate curve (straight line) by regression analysis, and obtains an inclination of the curve as a “downhill ground gear index”.

(5) The processing unit 41 of the main server 4 plots measurement data of each flat ground section to a horizontal direction speed-heart rate graph, obtains a horizontal direction speed-heart rate curve (straight line) by regression analysis, and obtains an inclination of the curve as a “flat ground gear index”.

(6) The mountaineering route is classified into the uphill ground, the flat ground, and the downhill ground here, but may not be classified. A gear index obtained without classification is simply referred to as a “gear index”.

### 1-9-6. Method of Calculating Slope Ground Adaptability Index

A method of calculating the slope ground adaptability index is as follows.

(1) The processing unit 41 of the main server 4 registers heart rates or the like of the user moving various mountaineering routes at each time in the database.

(2) The processing unit 41 of the main server 4 classifies (clusters) sections (or spots) of a statistical target route into an uphill ground, a flat ground, and a downhill ground based on the gradients (slope degrees) of the sections (or the spots). That is, the processing unit 41 clusters the measurement data of the spots in accordance with the gradients (the slope degrees). The statistical target route may be a partial route of a specific mountaineering route, may be a specific mountaineering route, may be a specific mountaineering route used for a specific period, or may be a whole mountaineering route.

(3) The processing unit 41 of the main server 4 plots measurement data of each uphill ground section to a gravity direction speed-heart rate graph, obtains a gravity direction speed-heart rate curve (straight line) by regression analysis, and obtains an inclination of the curve as a “slope ground adaptability index of an uphill ground”.

(4) The processing unit 41 of the main server 4 plots measurement data of each downhill ground section to a gravity direction speed-heart rate graph, obtains a gravity direction speed-heart rate curve (straight line) by regression analysis, and obtains an inclination of the curve as a “slope ground adaptability index of a downhill ground”.

(5) The mountaineering route is classified into the uphill ground, the flat ground, and the downhill ground here, but may not be classified. A slope ground adaptability index obtained without classification is simply referred to as a “slope ground adaptability index”.

### 1-9-7. Outputting Indexes

The processing unit 41 of the main server 4 outputs the calculated indexes to the user. For example, the processing unit 41 displays the indexes as a radar chart (which is a kind of graph) on the display unit 25 of the information terminal 2 (FIG. 13). Specifically, the processing unit 41 transmits image data for displaying the radar chart on the display unit 25 to the information terminal 2 of the user. The processing unit 21 of the information terminal 2 displays the radar chart on the display unit 25 based on the received image data.

The processing unit 41 of the main server 4 may display and superimpose previous indexes of the user and the same types of current indexes of the user on the same radar chart to make comparison. That is, in the system according to the embodiment, the plurality of indexes may include a plurality of indexes calculated for each period in which the user has exercised. In this case, the indexes can be compared by a period of the physical ability (or the balance of the physical ability). The comparison by the period is, for example, comparison between the plurality of physical abilities (balances) at temporally different previous times or comparison between the balances at previous and current times. The “current time” mentioned here may include a given period of time including the current time.

Similarly, in the system according to the embodiment, the plurality of indexes may include a plurality of indexes calculated for each gradient of the route along which the user exercises. In this case, comparison can be made by the gradient of the physical ability (or the balance). The comparison by the gradient is, for example, comparison by the uphill ground, the flat ground, and the downhill ground. In this case, it is possible to analyze a change in the physical ability (or the balance) which depends on the gradient of the route of the exercise.

The processing unit 41 of the main server 4 may superimpose and display the indexes of a certain user and the same types of indexes of another user on the same radar chart to make comparison (see FIG. 13). That is, in the system according to the embodiment, the number of users may be plural and the output unit may output a graph related to the indexes of the plurality of users. According to the graph, it is easy to compare the balances of the physical abilities of the plurality of users to each other.

The processing unit 41 of the main server 4 may superimpose and display the indexes between the users belonging to the same mountaineering party. Since the users belonging to the same mountaineering party move along the same mountaineering course at the same pace, it is easy to compare the physical abilities by only a variation in the heart rate. That is, in the system according to the embodiment, the plurality of users may be users in the same group. The same group is, for example, a group of users who all execute an exercise. For example, the same group is a mountaineering party moving together along the same route or a long-distance race team running along different sections of the same route in sequence. By comparing the physical abilities of the user in the group to each other and planning exercise content (route selection, role-sharing, and the like), it is possible to achieve an improvement in safety or performance of the whole group.

An example of an image design (radar chart) for comparing the indexes will be described below.

### 1-9-8. Specific Analysis

FIG. 5 is an exemplary graph illustrating a relation between an elapsed time and a heart rate for each user of the same mountaineering party. The horizontal axis of the graph represents an elapsed time and the vertical axis of the graph represents a heart rate [bpm].

Data plotted in a left region of the sheet surface of FIG. 5 is measurement data (here, data of a heart rate) acquired on an uphill ground of the mountaineering route, data plotted in the middle region is measurement data (here, data of the heart rate) acquired during a break, and data plotted in the right region is measurement data (here, data of the heart rate) acquired on a downhill ground. From the graph, it is generally difficult to evaluate the physical abilities of four individual users. Accordingly, the processing unit 41 of the main server 4 extracts a specific index from the measurement data of each user.

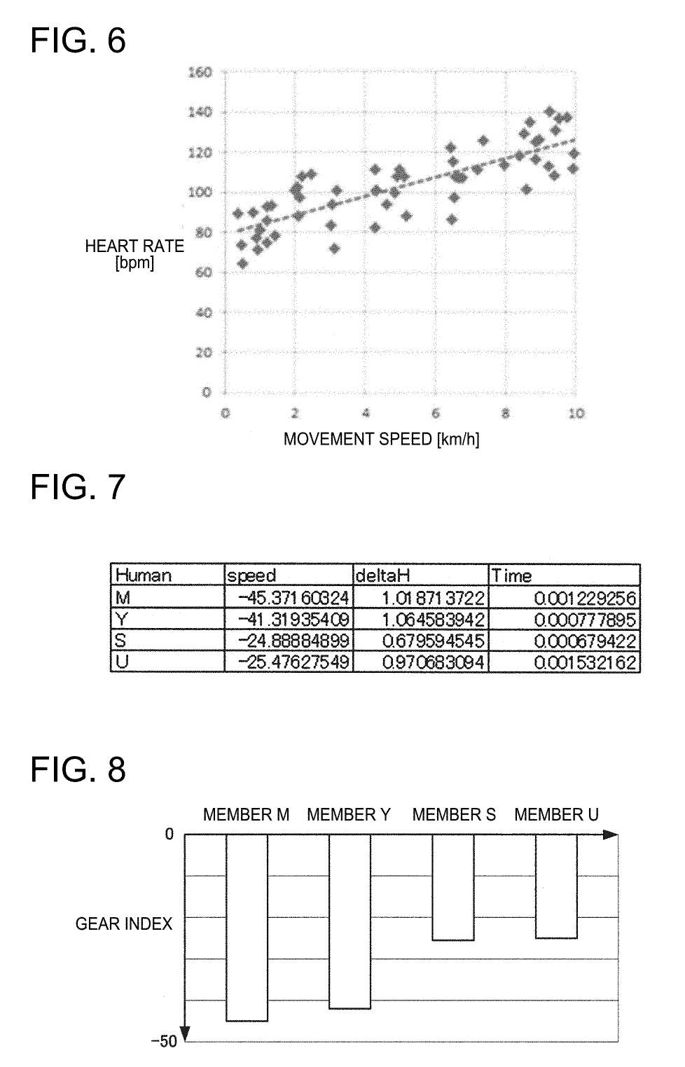

FIG. 6 is an exemplary graph illustrating a relation between a movement speed in the horizontal direction and a heart rate of a certain user. A dotted line indicates an example of a curve (regression line) obtained by regression analysis. The processing unit 41 of the main server 4 calculates a slope of the curve as the “gear index” of the user.

As illustrated in FIG. 6, it can be understood that as a movement speed in the horizontal direction is higher, the heart rate is higher. When the slope of the regression line is large, the gear index is high. It can be determined that as the gear index is higher, the physical ability is lower. The horizontal axis and the vertical axis of FIG. 6 are reversed. In this case, correlation between the gear index and the physical ability is reversed.

Similarly, the processing unit 41 of the main server 4 can generate a graph indicating a relation between the movement speed in the gravity direction and the heart rate and calculate the slope ground adaptability index from the slope of the curve shown in the graph.

Similarly, the processing unit 41 of the main server 4 can generate a graph indicating a relation between the elapsed time and the heart rate and calculate the stamina index from the slope of the curve shown in the graph.

FIG. 7 illustrates a table that shows the gear index (speed), the slope ground adaptability index (deltaH), and the stamina index (Time) for each user. Specifically, FIG. 7 illustrates regression analysis results of each user in regard to a change (speed) in the movement speed in the horizontal direction with respect to a change in the heart rate, the change deltaH in the movement speed in the gravity direction with respect to a change in the heart rate, and the elapsed time (exercise time) Time with respect to a change in the heart rate.

Here, each index is a slope of a linear regression line when the X axis represents a heart rate and the Y axis represents the movement speed of the horizontal direction, the movement speed in the gravity direction, or the elapsed time (exercise time). The X and Y axes can also be reversed. In this case, correlation between the index and the physical ability is reversed.

FIG. 8 is a diagram illustrating the gear index for each user. The gear index indicates an increase amount of the heart rate when the speed in the horizontal direction increases. In the example of FIG. 8, it can be understood that member M is a kind of member whose speed in the horizontal direction is not improved even when the heart rate increases as in a light automobile and member S is a reverse kind of member.

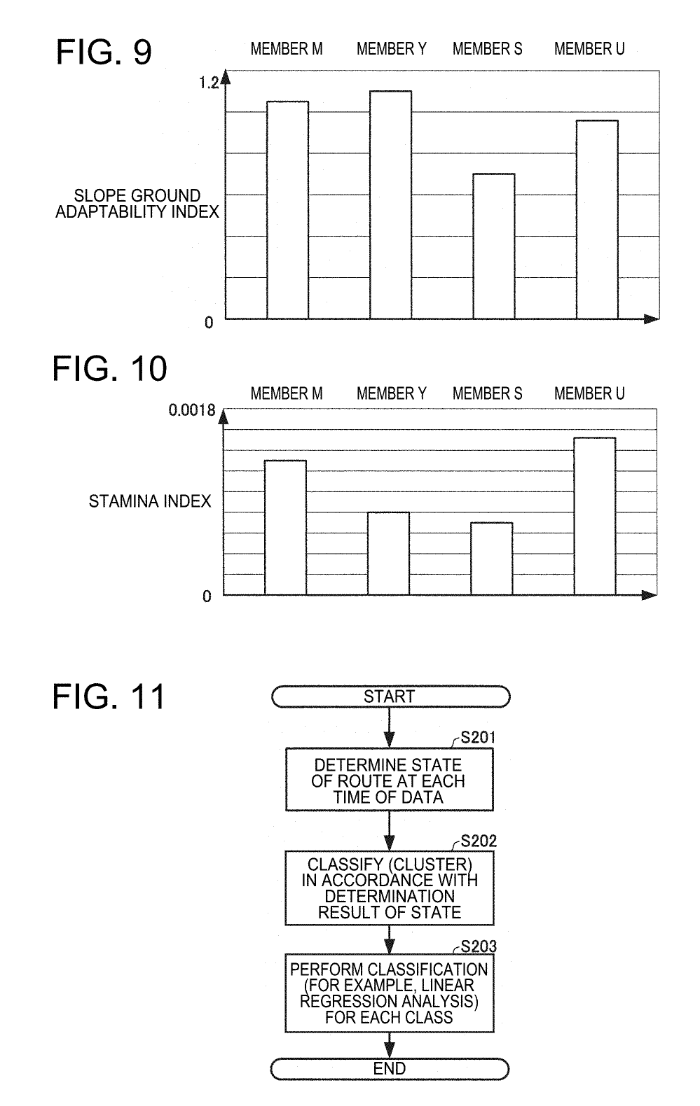

FIG. 9 is a diagram illustrating the slope ground adaptability index for each user. The slope ground adaptability index indicates an increase amount of the heart rate when the speed in the gravity direction increases. It can be understood that a member who has a higher numerical value of the slope ground adaptability index is a kind of member whose speed in the gravity direction may not be increased unless the heart rate considerably increases, that is, a kind of member week in an uphill ground.

FIG. 10 is a diagram illustrating the stamina index for each user. The stamina index indicates an increase amount of the heart rate over an elapsed time. It can be understood that a member who has a higher numerical value of the stamina index is a kind of member whose heart rate easily increases. That is, it can be understood that a member who has a higher stamina index consumes a larger energy consumption rate (fuel efficiency is poor and it is easy to be exhausted).

### 1-9-9. Flowchart (Individual)

FIG. 11 is an exemplary flowchart illustrating extraction of the indexes from measurement data of a certain user who has moved along a mountaineering route. Here, a case in which the measurement data includes information regarding the position of a user and information regarding a heart rate is assumed.

First, the processing unit 41 of the main server 4 determines a spot (or a section) state (gradient) which is a sampling source of individual measurement data based on data of the position included in the measurement data (S201).

Then, the processing unit 41 of the main server 4 classifies (clusters) sections (spots) of the mountaineering route using a determination result (S202). In the example of FIG. 5, the sections are classified into the first half of the mountaineering route=an uphill ground, an intermediate spot=a break spot, and the second half=a downhill.

Then, the processing unit 41 of the main server 4 calculates each index for each class by performing the regression analysis on the measurement data for each class. That is, the processing unit 41 of the main server 4 calculates an “uphill ground stamina index”, a “downhill ground stamina index”, a “flat ground stamina index”, an “uphill ground gear index”, a “downhill ground gear index”, a “flat ground gear index”, an “uphill ground slope ground adaptability index”, and a “downhill ground slope ground adaptability index”.

The processing unit 41 of the main server 4 may calculate the “stamina index”, the “gear index”, or the “slope ground adaptability index” by performing the regression analysis from the measurement data before the classification. That is, the processing unit 41 of the main server 4 may calculate an average “stamina index”, an average “gear index”, or an average “slope ground adaptability index” of the users in various route states instead of calculating the “stamina index”, the “gear index”, or the “slope ground adaptability index” for each route state.

### 1-9-10. Flowchart (Mountaineering Party)

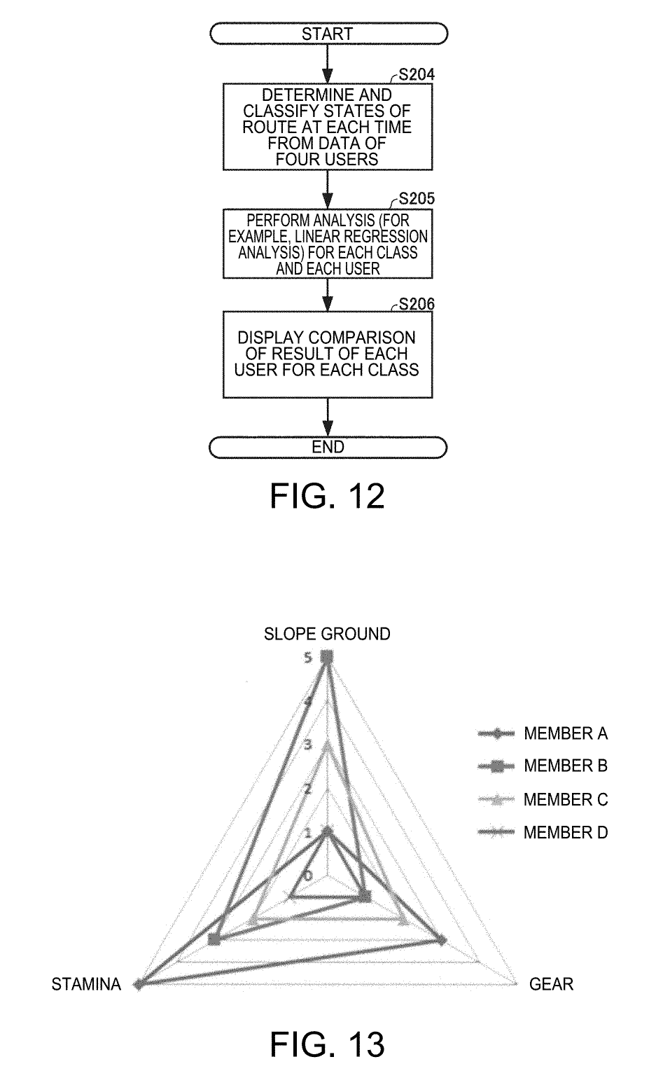

FIG. 12 is an exemplary flowchart illustrating extraction of the indexes from measurement data of four users belonging to the same mountaineering party.

First, the processing unit 41 of the main server 4 determines a state (gradient) of a spot (or a section) which is a sampling source of individual measurement data from the measurement data of four users (S204). Here, since four users belong to a common mountaineering party and all move, the fact that the users are considered to be located at the same spot for the same period of time is basically used (the details will be described below).

Then, the processing unit 41 of the main server 4 classifies (clusters) the measurement data in the sections (the spots) of the mountaineering route using the determination result and calculates the indexes for each class and each user (S205).

Then, the processing unit 41 of the main server 4 causes the display unit 25 of the information terminal 2 to display each index of each user in regard to at least one class in a format with which a difference between the users can be understood. A display destination of the indexes is, for example, the display unit 25 of the information terminal 2 used by at least one member among the four users, the display unit 170 of the electronic apparatus 1 used by at least one member among the four users, the display unit 25 of the information terminal 2 used by a user (coach) who manages the four users, or the display unit 170 of the electronic apparatus 1 used by the coach. Here, a display destination is assumed to be the display unit 25 of the information terminal 2.

### 1-9-11. Radar Chart

In the system according to the embodiment, the output unit may output a graph (here, which is assumed to referred to as a radar chart) related to a plurality of indexes (here, which is assumed to be the stamina index, the gear index, and the slope ground adaptability index). According to the radar chart, it is easy to intuitively grasp the balance of the physical abilities. Hereinafter, specific description will be made.

For example, the processing unit 41 causes the display unit 25 to display the plurality of indexes as a radar chart as in FIG. 13. At this time, image data for displaying the radar chart is transmitted from the processing unit 41 of the main server 4 to the processing unit 21 of the information terminal 2 via the communication unit 42, the network 3, and the communication unit 27.

In FIG. 13, three axes of the radar chart correspond to three types of indexes (the stamina index, the gear index, and the slope ground adaptability index). In the radar chart, the indexes of four users are distinguished from each other with mutually different colors, mutually different marks, or the like (the same applies to radar charts to be described below). Here, in the example of FIG. 13, each index is expressed at five stages. Each index can be expressed at five stages by performing scale conversion on a result (coefficient) of the above-described regression analysis. The direction of each axis of the radar chart (the positive or negative orientation of the index) is assumed to be set such that the physical ability increases far away from the center of the radar chart.

When the indexes of the plurality of users are plotted on one radar chart in this way, the physical ability related to the stamina index can be compared between the users, the physical ability related to the slope ground adaptability index can be compared between the users, and the physical ability related to the gear index can be compared. In the example of FIG. 13, a case is illustrated in which member A has the highest physical ability related to the stamina index, member B has the highest physical ability related to the slope ground adaptability index, and member A has the highest physical ability related to the gear index.

According to the radar chart, it is possible to determine which physical ability of the same user is excellent among the physical ability related to the stamina index, the physical ability related to the slope ground adaptability index, and the physical ability related to the gear index. In the example of FIG. 13, it can be understood that member A is a person of “stamina type” in which the physical ability related to the stamina index is excellent, member B is a person of “slope ground type” in which the physical ability related to the slope ground adaptability index is excellent, member C is a person of “slope ground type” in which the physical ability related to the slope ground adaptability index is excellent, and member D is a person of a type in which all the physical abilities are uniform.

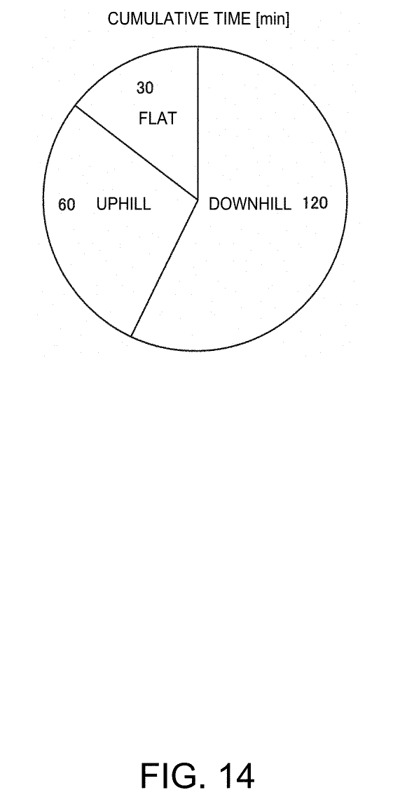

Here, in the system according to the embodiment, there is a difference in the number of pieces of measurement data (the number of samples) in accordance with a class (distinction of an uphill ground, a flat ground, a downhill ground, a break spot). Therefore, when the indexes are calculated for each class, calculation precision of the index is considered to be also different in accordance with the class. Accordingly, when the indexes are calculated for each class, the processing unit 41 of the main server 4 may display the number of samples of the measurement data in each class, as illustrated in, for example, FIG. 14.

FIG. 14 illustrates an example of a circular graph that shows the number of samples of each class as a cumulative time. At this time, image data for displaying the circular graph is transmitted from the processing unit 41 of the main server 4 to the processing unit 21 of the information terminal 2 via the communication unit 42, the network 3, and the communication unit 27.

In the example of FIG. 14, the cumulative time of the uphill ground is 120 minutes, the cumulative time of the downhill ground is 60 minutes, and the cumulative time of the flat ground is 30 minutes. When a sampling period of the measurement data is constant, a ratio of the numbers of samples is equal to a ratio of the cumulative times. Accordingly, in the example of FIG. 14, it can be understood that the number of samples of the measurement data related to the uphill ground is the largest, that is, precision of the index of the uphill ground is the highest. Here, the cumulative time of each class is displayed, but a cumulative distance may be displayed instead of the cumulative time.

### 1-9-12. Data of Gradient

In the system according to the embodiment, the data of the mountaineering route may include gradient information. The gradient information may be information for calculating a gradient, may be information regarding a gradient, may be another piece of information regarding the uphill ground, the flat ground, or the downhill ground derived from the gradient. In this case, it is possible to analyze a change in the physical ability which depends on a gradient of the route along which an exercise is executed. For example, it is possible to determine whether the user can exert the physical ability in a slope ground or a flat ground. Hereinafter, the gradient information is assumed to be calculated appropriately and added to the data of the mountaineering route by the processing unit 41 of the main server 4.

Here, when the sections (the spots) of the mountaineering route are clustered into the uphill ground, the flat ground, the downhill ground, and the break spot, it is necessary to measure a gradient of a spot at which the user is walking or running. Therefore, several methods of measuring a gradient will be described.

### 1-9-13-1. Method Using Positional Information

First, a method of measuring a gradient using positional information will be described.

The positional information mentioned here is information included in the measurement data and is information indicating 3-dimensional positional coordinates (λ, ϕ, h) of the user (λ: latitude, ϕ: longitude, h: altitude). The positional coordinates are calculated, for example, based on an output of the GPS sensor 110 of the processing unit 120 of the electronic apparatus 1 and are one piece of measurement data transmitted to the information terminal 2.

The processing unit 41 of the main server 4 calculates a gradient grad using the altitude h based on an output of the GPS sensor 110. The gradient grad is calculated using the spot and the following calculation equation in which positional coordinates (λ1, ϕ1, h1) and (λ2, ϕ2, h2) of spots before and after the spot.

$$grad = \frac{{h}_{2} - {h}_{1}}{d ( ( {λ}_{1} , {φ}_{1} ) , ( {λ}_{2} , {φ}_{2} ) )}$$

Here, the denominator “d(A, B)” of the equation is a distance function of calculating a horizontal distance between two points A and B.

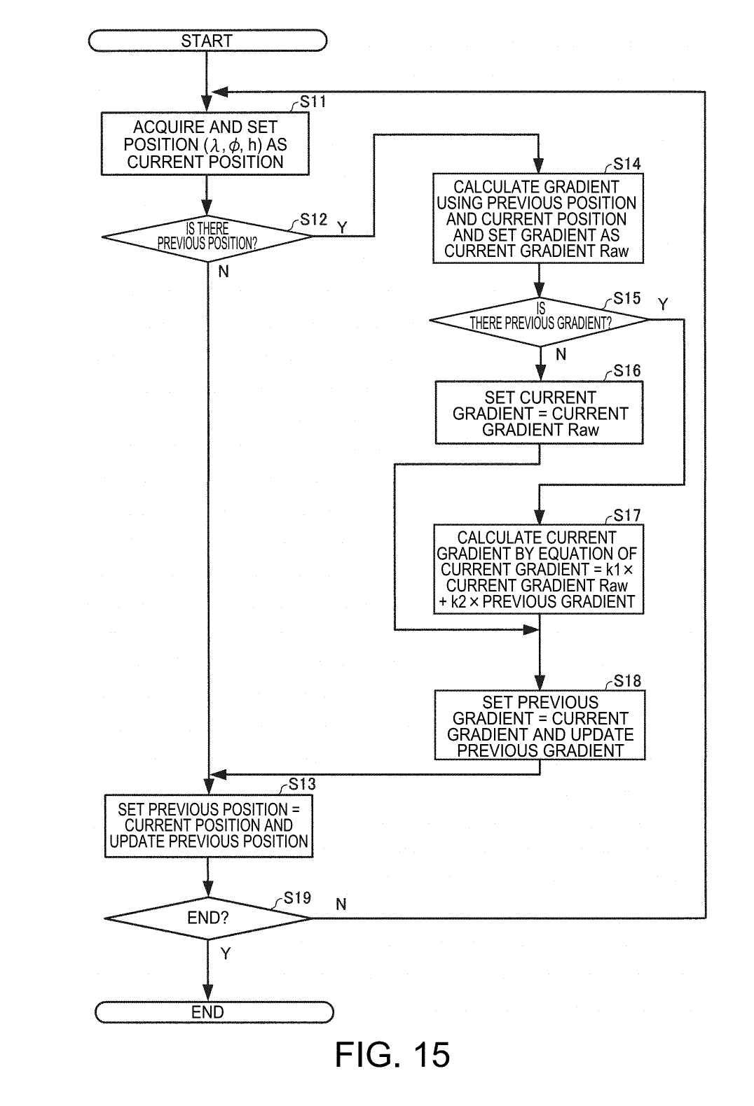

FIG. 15 is an exemplary flowchart illustrating a process of calculating a gradient based on positional information. The flowchart is performed whenever positional information is acquired. Here, a case is assumed in which measurement data including the positional information is transmitted in sequence during mountaineering from the electronic apparatus 1 to the main server 4 via the information terminal 2. That is, the flow of the FIG. 15 is a flow in which gradients of spots of the mountaineering route can be measured in sequence (mostly, the flow can also be executed after the mountaineering).

The processing unit 41 of the main server 4 stores the positional coordinates (λ, ϕ, h) as a current position in the storage unit 44 when the positional coordinates (λ, ϕ, h) are acquired (S11).

Then, the processing unit 41 determines whether there is the previous position in the storage unit 44 (S12). When the positional coordinates have not been acquired even once or an elapsed time from a storage time of a previous position is a given time or more (for example, 5 minutes or more), the processing unit 41 may determine that there is no previous position.

When there is no previous position in the storage unit 44 (N in S12), the processing unit 41 stores the current position acquired in step S11 as the previous position in the storage unit 44 (S13) and ends the flow (waits until the positional information is updated subsequently).

Conversely, when there is the previous position in the storage unit 44 (Y in S12), the processing unit 41 calculates a current gradient using the previous position and the current position (S14).

Subsequently, the processing unit 41 determines whether there is an effective previous gradient (S15). When the gradient has not been acquired even once or an elapsed time from a storage time of a previous gradient is a given time or more (for example, 5 minutes or more), the processing unit 41 may determine that there is no effective previous gradient. The processing unit 41 stores the calculated gradient as a current gradient Raw (S16).

When there is no effective previous gradient (N in S15), the processing unit 41 stores the current gradient Raw as a current gradient in the storage unit 44 without change (S16) and treats the current gradient as a gradient at the final current time.

Conversely, when there is the effective previous gradient (Y in S15), the processing unit 41 calculates the current gradient using the current gradient Raw and the previous gradient (S17). At this time, scalars k1 and k2 are used and a calculation equation of the current gradient=(k1×the current gradient Raw)+(k2×the previous gradient) is used.

Here, the scalars k1 and k2 satisfy equations of 0≤k1, k2≤1, and k1+k2=1. The calculation equations form a simple lowpass filter. The reason for performing the filter is because a variation in the positional information (altitude) output by the GPS sensor is absorbed.

Then, the processing unit 41 substitutes a value of the calculated current gradient to the previous gradient (S18) and the process proceeds to the process of updating the previous position (S13).

Then, the processing unit 41 determines end (S19). When the end is determined (Y in S19), the flow ends. Otherwise (N in S19), the process returns to the initial process (S11).

### 1-9-13-2. Method Using Atmospheric Pressure Information

Next, a method of measuring a gradient using atmospheric information will be described.

The atmospheric pressure information mentioned herein is information included in the measurement data, is calculated based on an output of the pressure sensor 112 by the processing unit 120 of the electronic apparatus 1, and is one of the measurement data transmitted to the information terminal 2.

The processing unit 41 of the main server 4 calculates an altitude using the atmospheric pressure and calculate a gradient. Here, an example of a case in which an output of the GPS sensor 110 is not used will be described.

In the method using the positional information, a “vertical movement amount per horizontal unit distance” has been calculated as a gradient. Here, a “vertical movement amount per unit time” is calculated as a gradient.

The magnitude of the unit time may have any value. For example, the unit time is assumed to be “1 minute”. The gradient grad is calculated using, for example, the altitude h calculated by the following calculation equation.

$$h = \frac{( {( \frac{{P}_{0}}{P} )}^{\frac{1}{5.257}} - 1 ) \times ( T + 273.15 )}{0.0065}$$

Here, P is an atmospheric pressure at a current spot, P₀ is a sea level pressure, and T is a temperature.

Therefore, to calculate the altitude h, not only a numerical value of the atmospheric pressure P but also the sea level pressure P₀ and the temperature T are actually necessary.

The temperature T is information included in the measurement data, is calculated based on an output of the temperature sensor 116 by the processing unit 120 of the electronic apparatus 1, and is one of the measurement data transmitted to the information terminal 2. A disposition destination of the temperature sensor 116 is preferably near the pressure sensor 112.

A method of determining the sea level pressure P₀ differs for each electronic apparatus 1. For example, there is a method of obtaining the sea level pressure P₀, for example, through backward calculation from an altitude based on an output of the GPS sensor 110 or a method of acquiring the information from the main server 4 or the weather server 5.

In either method, a method of calculating a vertical movement amount per unit time from a plurality of altitudes obtained from an output of the pressure sensor 112 at each time is adopted.

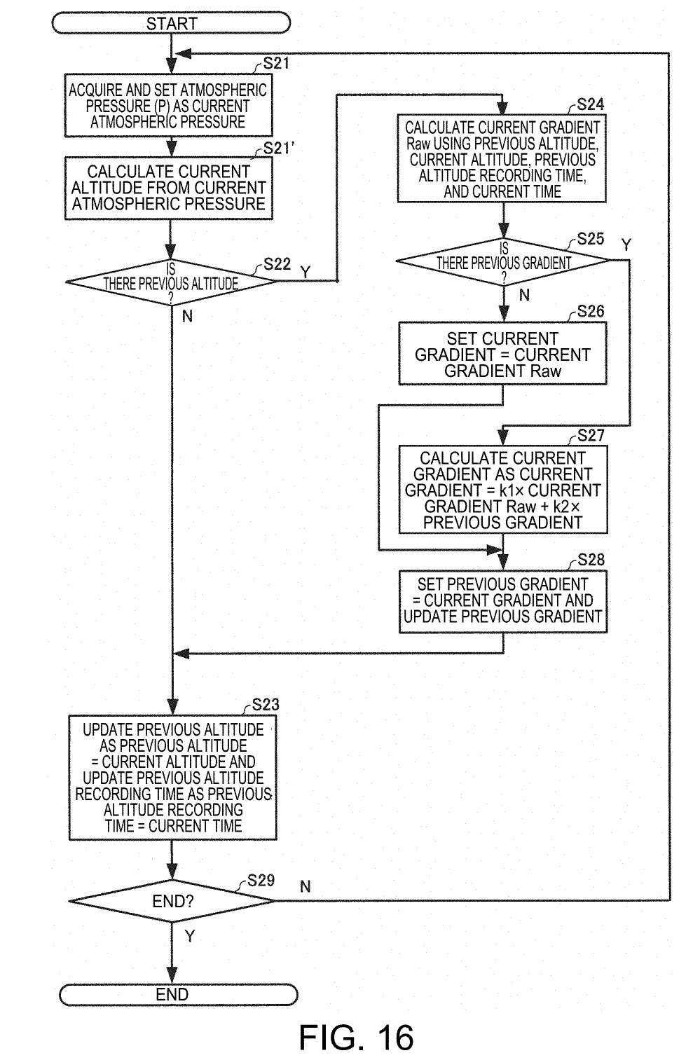

FIG. 16 is an exemplary flowchart illustrating a process of calculating a gradient based on weather information. The flowchart is performed whenever atmospheric pressure information is acquired. Here, a case is assumed in which measurement data including the storage information is transmitted in sequence during mountaineering from the electronic apparatus 1 to the main server 4 via the information terminal 2. That is, according to the flow of the FIG. 16, gradients of spots of the mountaineering route can be measured in sequence.

Here, differences between FIGS. 15 and 16 will be mainly described and common points will be described simply. The flow illustrated in FIG. 16 is substantially the same as the flow illustrated in FIG. 15, but the flow illustrated in FIG. 16 is different in that only an atmospheric pressure is used as an actually measured value.

The processing unit 41 of the main server 4 stores the atmospheric pressure (P) as a current atmospheric pressure in the storage unit 44 when the atmospheric pressure (P) is acquired (S21).

Then, the processing unit 41 converts the current atmospheric pressure into an altitude (S21′).

Then, the processing unit 41 determines whether there is a previous altitude in the storage unit 44 (S22). When the atmospheric pressure has not been acquired even once or an elapsed time from a storage time of the previous altitude is a given time or more (for example, 5 minutes or more), the processing unit 41 may determine that there is no previous altitude.

When there is no previous altitude in the storage unit 44 (N in S22), the processing unit 41 stores the current altitude acquired in step S21′ as the previous altitude in the storage unit 44 (S23) and further also store a previous altitude recording time similarly and ends the flow (waits until the atmospheric pressure information is updated subsequently).

Conversely, when there is the previous altitude in the storage unit 44 (Y in S22), the processing unit 41 calculates a current gradient using the previous altitude, the current altitude, the previous altitude recording time, and the current time (S24).

Subsequently, the processing unit 41 determines whether an effective previous gradient (S25). When the gradient has not been acquired even once or an elapsed time from a storage time of a previous gradient is a given time or more (for example, 5 minutes or more), the processing unit 41 may determine that there is no effective previous gradient. The processing unit 41 stores the calculated gradient as a current gradient Raw (S26).

When there is no effective previous gradient (N in S25), the processing unit 41 stores the current gradient Raw as a current gradient in the storage unit 44 without change (S26) and treats the current gradient as a gradient at the final current time.

Conversely, when there is the effective previous gradient (Y in S25), the processing unit 41 calculates the current gradient using the current gradient Raw and the previous gradient (S27). At this time, scalars k1 and k2 are used and a calculation equation of the current gradient=(k1×the current gradient Raw)+(k2×the previous gradient) is used.

Here, the scalars k1 and k2 satisfy equations of 0≤k1, k2≤1, and k1+k2=1. The calculation equations form a simple lowpass filter. The reason for performing the filter is because a variation in the atmospheric pressure information is absorbed.

Then, the processing unit 41 substitutes a value of the calculated current gradient to the previous gradient (S28) and the process proceeds to the process of updating the previous altitude (S23).

Then, the processing unit 41 determines end (S29). When the end is determined (Y in S29), the flow ends. Otherwise (N in S29), the process returns to the initial process (S21).

### 1-9-13-3. Method Using Pressure Difference Between Two Points

Further, the processing unit 41 may use a method of directly obtaining an altitude difference from an atmospheric pressure difference between two points. For example, the following calculation equation may be used.

$$Δ h = \frac{{R}_{d} {T}_{m}}{g} \cdot \frac{Δ p}{{p}_{m}}$$

Here, Rd is a gas constant of dry air, Tm is an average temperature between two points, and pm is an average atmospheric pressure between two points. In this case, since the altitude calculation is not performed at each time, there is an advantage of simplifying the process (reducing a processing load). An example of a flow in this case will be described below.

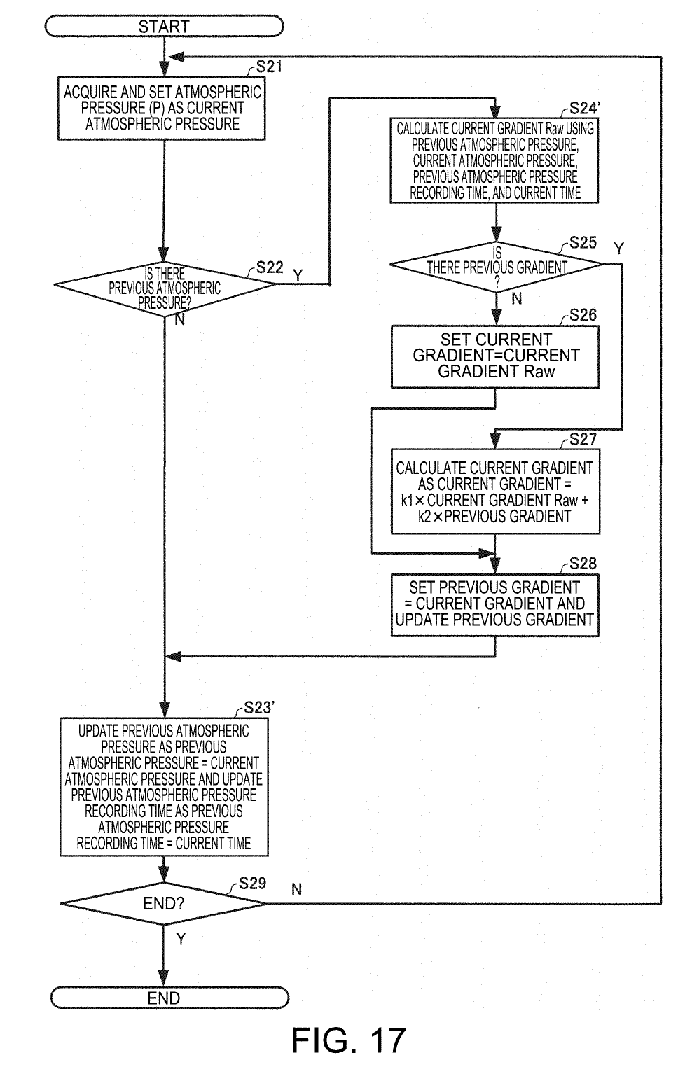

FIG. 17 is an exemplary flowchart illustrating a process of calculating a gradient based on an atmospheric difference between two points (a process performed when the positional information is acquired).

The processing unit 41 of the main server 4 stores the atmospheric pressure (P) as a current atmospheric pressure in the storage unit 44 when the atmospheric pressure (P) is acquired (S21).

Then, the processing unit 41 determines whether there is a previous atmospheric pressure in the storage unit 44 (S22). When the atmospheric pressure has not been acquired even once or an elapsed time from a storage time of the previous atmospheric pressure is a given time or more (for example, 5 minutes or more), the processing unit 41 may determine that there is no previous atmospheric pressure.

When there is no previous atmospheric pressure in the storage unit 44 (N in S22), the processing unit 41 stores the current atmospheric pressure acquired in step S21 as the previous atmospheric pressure in the storage unit 44 (S23′) and further also store a previous atmospheric pressure recording time similarly and ends the flow (waits until the atmospheric pressure information is updated subsequently).

Conversely, when there is the previous position in the storage unit 44 (Y in S22), the processing unit 41 calculates a current gradient using the previous atmospheric pressure, the current atmospheric pressure, the previous atmospheric pressure recording time, and the current time (S24′).

Subsequently, the processing unit 41 determines whether an effective previous gradient (S25). When the gradient has not been acquired even once or an elapsed time from a storage time of a previous gradient is a given time or more (for example, 5 minutes or more), the processing unit 41 may determine that there is no effective previous gradient. The processing unit 41 stores the calculated gradient as a current gradient Raw (S26).

When there is no effective previous gradient (N in S25), the processing unit 41 stores the current gradient Raw as a current gradient in the storage unit 44 without change (S26) and treats the current gradient as a gradient at the final current time.

Conversely, when there is the effective previous gradient (Y in S25), the processing unit 41 calculates the current gradient using the current gradient Raw and the previous gradient (S27). At this time, scalars k1 and k2 are used and a calculation equation of the current gradient=(k1×the current gradient Raw)+(k2×the previous gradient) is used.

Here, the scalars k1 and k2 satisfy equations of 0≤k1, k2≤1, and k1+k2=1. The calculation equations form a simple lowpass filter. The reason for performing the filter is because a variation in the atmospheric pressure information is absorbed.

Then, the processing unit 41 substitutes a value of the calculated current gradient to the previous gradient (S28) and the process proceeds to the process of updating the previous atmospheric pressure (S23′).

Then, the processing unit 41 determines end (S29). When the end is determined (Y in S29), the flow ends. Otherwise (N in S29), the process returns to the initial process (S21).

### 1-9-13-4. Method of Using Data of Mountaineering Route

Here, when the data of the mountaineering route is included in advance in the mountaineering plan data, the gradient can be calculated based on the data of the mountaineering route.

Here, when information regarding the altitude is not included in the data of the mountaineering route, the processing unit 41 of the main server 4 necessarily accesses another server (map server) via the network 3 and acquire the information regarding the altitude of the mountaineering route. The map server is a server that stores information regarding 3-dimensional positional coordinates of each spot and has a function of supplying information regarding the positional coordinates of a designated area in response to a request from another server or an information terminal.

### 1-9-14. Function of Excluding Break Section

The processing unit 41 of the main server 4 may detect a section corresponding to a break (that is, measurement data acquired during a break) from the measurement data related to the mountaineering route. Whether the user takes a break can be determined based on data of a position, data of a speed, data of acceleration, and data of an angular velocity included in the measurement data. Whether the user takes a break can also be determined in sequence during a period in which the user is executing the mountaineering.

For example, the processing unit 120 of the electronic apparatus 1 monitors whether the user stops during the mountaineering using a stop determination technology of applying a speed which is based on an output of the GPS sensor 110 or a change in a position which is based on an output of the GPS sensor 110 or a method of monitoring a body motion which is based on an output of the acceleration sensor 113. When it is determined that the user stops, identification information indicating the stop (a stop flag) may be added to the measurement data acquired at that time.

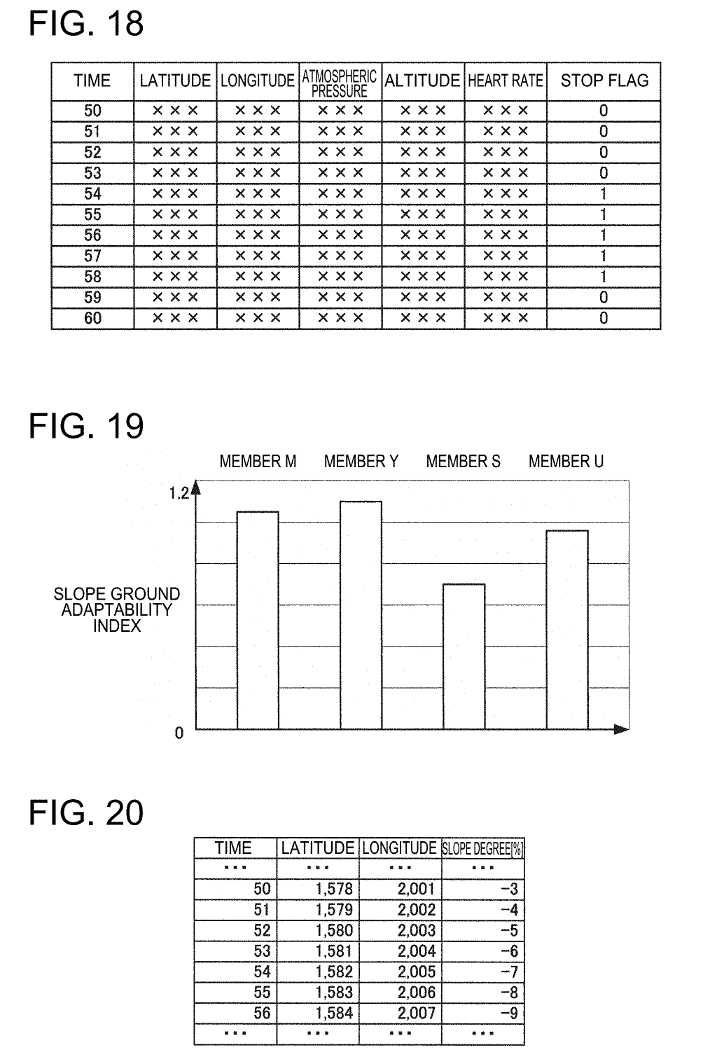

For example, a format illustrated in FIG. 18 can be used as a format of the measurement data. The stop flag is added to individual measurement data. A status (ON/OFF) of the stop flag added to the measurement data indicates whether the user stops at the time of acquiring the measurement data.

When the stop flag is added to the individual measurement data in this way, the processing unit 41 of the main server 4 can improve calculation precision of each of the index related to the uphill ground, the index related to the flat ground, and the index related to the downhill by excluding the measurement data corresponding to the stop of the user from a target of the above-described regression analysis.

Alternatively, the processing unit 41 of the main server 4 can reliably calculate the index related to the break (the stamina index during the break) by setting only the measurement data corresponding to the stop as a target of the regression analysis.

### 1-9-15. Comparison Between Users (in Mountaineering Party)

The above-described indexes have meanings by comparing the users or the indexes. Therefore, how the comparison is made is important.

For example, there is a method of comparing users belonging to the same mountaineering party. Incidentally, comparison between the users belonging to the same mountaineering party can be applied as in comparison between users participating in the same competition.

Since a mountaineering course along which the users move is common to the users belonging to the same mountaineering party, for example, comparison as in FIG. 19 is effective. Since FIG. 19 is the same as the graph described in FIG. 9, the description thereof will be omitted here.

The result of the above-described classification (clustering) is also the same between the users belonging to the same mountaineering party. Therefore, when the measurement data of any user is clustered, there is the advantage that it is not necessary to cluster the measurement data of the other users. That is, since a section in which a first user belonging to the same mountaineering party is located for a certain period of time is substantially the same as a section in which a second user belonging to the same mountaineering party is for the same period of time, a class to which the section in which the first user is located belongs and a class to which the second user is located belongs can be considered to be the same. That is, when the former section is an “uphill ground”, the latter section is also the “uphill ground”. Here, a case in which a part of the mountaineering route is clustered for each section will be described. However, it is regardless to say that the part of the mountaineering route can be clustered for each spot.

### 1-9-16. Error Between Electronic Apparatuses

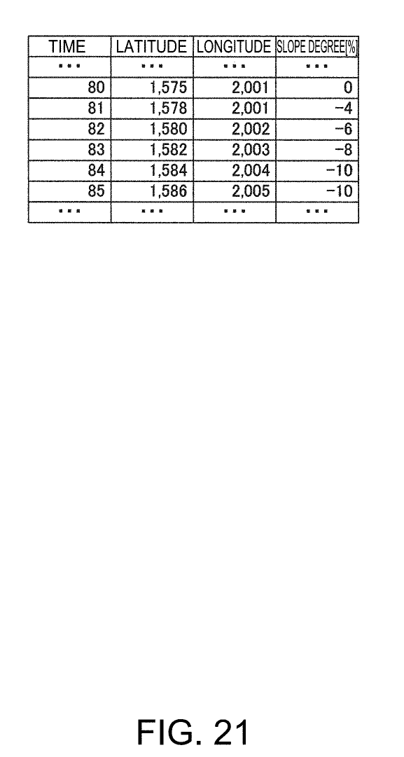

FIG. 20 is a diagram illustrating an example of measurement data acquired from the first user (a first electronic apparatus 1) along a certain route. FIG. 21 is a diagram illustrating an example of measurement data acquired from the second user (a second electronic apparatus 1) along the same route.

As illustrated in FIGS. 20 and 21, even when the first and second users walk along the same mountaineering route, at least a part (for example, a slope degree) of the measurement data differ between the users due to an error of a sensor of the electronic apparatus 1 even in the measurement data of the same section in some cases. Here, the slope degree is an amount equivalent to a gradient. In the present specification, the slope degree and the gradient are used as the same meaning (both the slope degree and the gradient indicate the degrees of a slope of the route).

When an error of the sensor occurs in this way and, for example, an index related to a section with slope degrees of +5% and an index related to a section with slope degrees of +7% are compared as the same type of index, reliability of a comparison result may considerably deteriorate.

Accordingly, even when the processing unit 41 of the main server 4 calculates information (for example, a gradient) related to the mountaineering route from the measurement data of the user of the first electronic apparatus 1, a method of calculating the same information (for example, a gradient) from the measurement data of the user of the second electronic apparatus 1 independently from the first electronic apparatus 1 and comparing both the pieces of information to each other (suppressing the error by averaging the information) may be adopted. Hereinafter, a method of managing a common slope degree based on the measurement data of two users will be proposed.

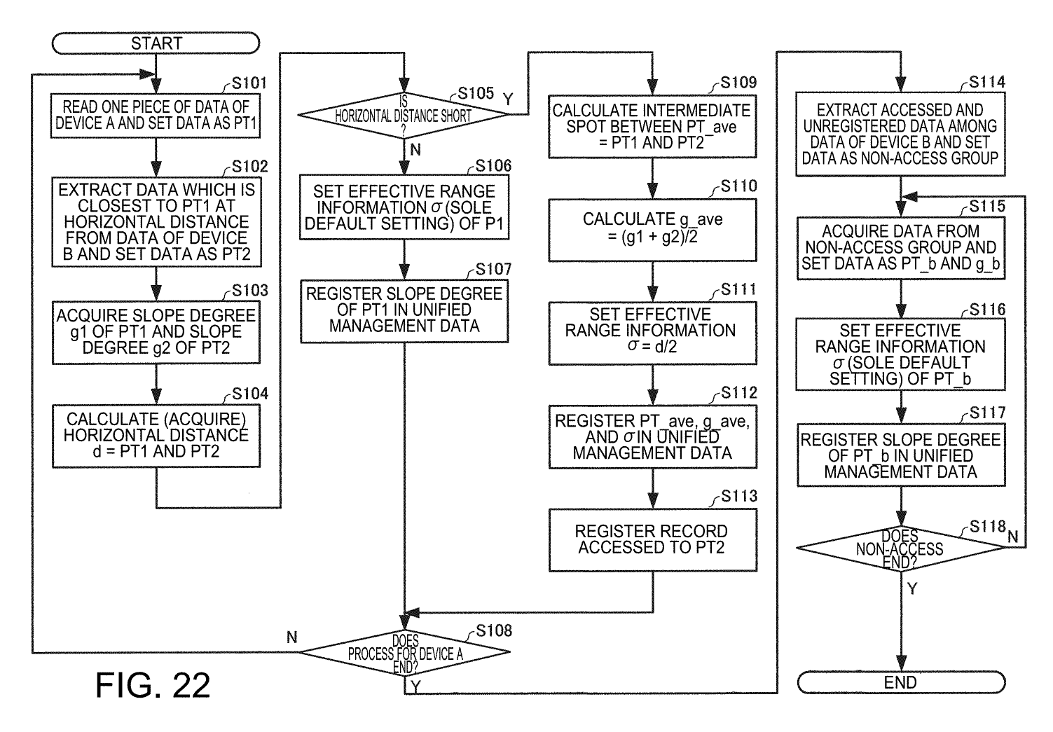

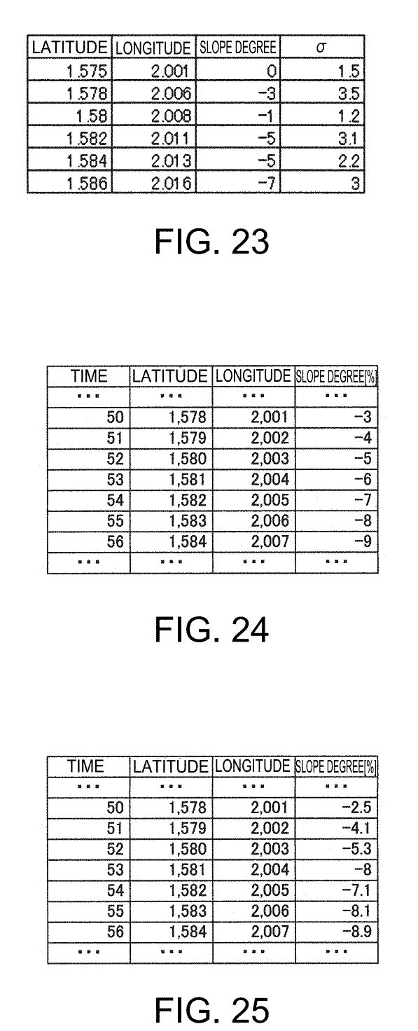

FIG. 22 is an exemplary flowchart illustrating a process of managing a common slope degree based on the measurement data acquired from two different users (the first and second electronic apparatuses 1). FIG. 23 is an exemplary table illustrating data of the slope degrees (data indicating topography) acquired through the process of FIG. 22. The data (which is a table and is also referred to as unified management data) illustrated in FIG. 23 is stored in, for example, the storage unit 44. The data stored in the storage unit 44 includes a value of a slope degree at each positional coordinates (latitude and longitude) in the horizontal direction and a variation σ (effective range information). The variation σ (effective range information) is stored to be used to determine an effective range of the data.

A flow of FIG. 22 will be described. Hereinafter, the first electronic apparatus 1 of the first user is referred to as a “device A” and the second electronic apparatus 1 of the second user is referred to as a “device B”. The first and second users are users (users belonging to the same mountaineering party) who move together along the same mountaineering route.

First, the processing unit 41 of the main server 4 accesses data PT1 of the positional coordinates among the measurement data related to the device A (S101). When the process of step S101 is not a process performed for the first time, an access destination of step S101 is increased by one from the immediately previous access destination (reading proceeds) (S101).

Subsequently, the processing unit 41 accesses the data of the positional coordinates among the measurement data related to the device B, retrieves data which is the closest to the data PT1, and set the detected data as PT2 (S102). Subsequently, the processing unit 41 calculates a slope degree g1 of the data PT1 and a slope degree g2 of the data PT2 (S103).

Subsequently, the processing unit 41 acquires a distance (a horizontal distance d) between the data PT1 and the data PT2 in the horizontal direction (S104). The horizontal distance d may be calculated as necessary.

Subsequently, the processing unit 41 determines the horizontal distance d is a given distance or less (for example, 10 m or less) (S105).

When d is the given distance or more (for example, 10 m or more) (N in S105), the processing unit 41 determines that there is no data of the device B matching the data PT1 and registers topographic data (latitude, longitude, and altitude) which is based on the data PT1 solely in the foregoing table (S106 and S107). At this time, a set value of a may be used as a default set value or may be set in accordance with a distance between the pieces of data before and after the data PT1 stored in the device A.

Conversely, d is the given distance or less (for example, 10 m or less) (Y in S105), the processing unit 41 calculates unified topographic data (latitude, longitude, and slope degree) by mixing (averaging) the positional coordinates and registers the unified topographic data in the foregoing table (S109 to S113). AT this time, the set value of the variation σ (the effective range information) may be changed in accordance with a distance (S111). When the data PT1 and the PT2 are mixed and registered, the processing unit 41 writes a record accessed to the data PT2 for a subsequent process (S113).

When the process ends on all the data PT1 of the device A related to the mountaineering route (Y in S108), the data of the device A is all registered in the unified topographic data.

Thereafter (Y in S108), the processing unit 41 registers data unregistered in the unified topographic data among the data of the device B as sole information in the topographic data (S114 to S118).

Here, whether the data is not registered in the unified topographic data is determined in accordance with whether there is an accessed record performed in the front-stage process (S113) (S114). By registering only information for which there is no accessed record in the unified topographic data (S115 to S117), it is possible to manage all the information in a unified manner.

The processing unit 41 performs the processes (S114 to S117) on all the data PT2 of the device B related to the mountaineering route (N in S118). When the processes are completed on all the data (Y in S118), the flow ends.

In the foregoing flow, the steps may be permutated in order within a possible range.

Here, the data of the positional coordinates has been compared between the first and second users, but temporal data may be compared between the first and second users. The comparison between the data of the positional coordinates can be made with a simpler process than the comparison of the temporal data.

An example of a case in which there are two pieces of data of the devices A and B is illustrated in FIGS. 24 and 25.

When the first and second users are proven to execute the same behavior in advance (that is, the users are located at the same spot at the same time), a method of comparing the data of the positional coordinates at the common time and registering an average of the data can be adopted. For example, in the examples of FIGS. 24 and 25, the topographic data at time 50 can be calculated by the following equations.

Average latitude=(1.578+1.578)/2=1.578

Average longitude=(2.001+2.001)/2=2.001

Average slope degree=(−3+(−2.5))/2=−2.75

### 1-9-17. Comparison Between Periods of Same User

The processing unit 41 of the main server 4 may compare the indexes at different periods of the same user. For example, the processing unit 41 compares measurement data (or the indexes) at the time of movement of a certain mountaineering route by the user to measurement data at the time of movement of the same mountaineering route by the user at a different period.

In this case, data acquired in advance as the topographic data illustrated in FIG. 23 can be used. By using the acquired topographic data, it is possible to compare the data more accurately.

When indexes are calculated in regard to a route along which the user has not moved previously, the processing unit 41 of the main server 4 may perform a process of newly registering the topographic data of the route.

### 1-9-17-1. Matching Routes

The processing unit 41 of the main server 4 determines which mountaineering route is the same as a new mountaineering route among the mountaineering routes registered in the database based on a distance between a movement trajectory of the user at the time of movement of the new mountaineering route and the registered mountaineering route. The distance between the new movement trajectory and the registered mountaineering route (a previous movement trajectory) or a distance between a spot at which the user is located and the registered mountaineering route (the previous movement trajectory) can be calculated using the following equations.

$${L}_{WP 3 \_Data 1} = {min}_{1 \leq i \leq {N}^{( 1 )}} \{ dist ( ( {λ}_{i}^{( 1 )} , {φ}_{i}^{( 1 )} ) , ( {λ}^{( WP 3 )} , {φ}^{( WP 3 )} ) ) \}$$

$${L}_{Data 3 \_Data 4} = {max}_{1 \leq i \leq {N}^{( 3 )}} \{ {min}_{1 \leq j \leq {N}^{( 4 )}} \{ dist ( ( {λ}_{i}^{( 3 )} , {φ}_{i}^{( 3 )} ) , ( {λ}_{j}^{( 4 )} , {φ}_{j}^{( 4 )} ) ) \} \}$$

For example, the processing unit 41 of the main server 4 determines whether a distance calculated with the calculation equations is a given distance or less (for example, 10 m or less). When the distance is the given distance or less, the new mountaineering route is considered to be the same as the registered mountaineering route and a subsequent process is performed.

The processing unit 41 of the main server 4 (or the processing unit 21 of the electronic apparatus 1) determines that the same mountaineering route as the mountaineering route along which the user is moving or has moved is registered in the database, the processing unit 41 may automatically compare the measurement data (or the indexes) related to the registered mountaineering route to the current measurement data (or the indexes) and supply the user with a display screen for checking a previous condition and a current condition, a display screen for checking a previous condition and a current physical ability, or the like. A display destination of the display screen is the display unit 25 of the information terminal 2 (the display unit 170 of the electronic apparatus 1).

When only partial sections of the current mountaineering route are the same as those of the registered mountaineering route, the processing unit 41 of the main server 4 may consider that substantially the same mountain regions and set the mountain regions as comparison targets.

### 1-9-18. Clustering by Weather

In the system according to the embodiment, the plurality of indexes (which are assumed to be the stamina index, the gear index, the slope ground adaptability index here) may include the plurality of indexes (which are assumed to be the stamina index, the gear index, the slope ground adaptability index here) calculated for each weather of a route along which the user exercises. In this case, comparison between balances of the physical abilities by weather can be made. The comparison by weather is, for example, comparison by a fair sky, a rainy sky, a cloudy sky or comparison by atmospheric pressure. In this case, it is possible to analyze a change in the balance which depends on weather of the route of the exercise. Hereinafter, weather information is assumed to be appropriately acquired and added to the data of the mountaineering route by the processing unit 41 of the main server 4.

In this case, the system according to the embodiment can classify (cluster) the measurement data of the spots of the mountaineering route in accordance with the gradients (slope degrees) of the spots and can also cluster the measurement data by weather information (obtained from an atmospheric pressure).

For example, the processing unit 41 of the main server 4 acquires weather information (bad weather, normal weather, and fine weather) by performing atmospheric pressure calibration at a stage of post-processing (after-processing) at a time at which the measurement data of the mountaineering route is obtained.

Specifically, when an altitude of a certain spot is calculated from an atmospheric pressure of the spot, the processing unit 41 uses the atmospheric pressure P₀ of the spot at 0 m above the sea level, and thus adds the value of the atmospheric pressure P₀ to the measurement data (measurement data of each spot). Then, by clustering the measurement data of the spots in accordance with a range to which the value of P₀ belongs and calculating the above-described indexes for each class, it is possible to analyze a change in the physical ability by weather. Accordingly, for example, the user can grasp his or her physical ability which depends on weather, such as “strong to low atmospheric pressure” or “strong to high atmospheric pressure”.

Since the method using the atmospheric pressure P₀ is easily affected by calibration precision, the processing unit 41 may adopt a method of directly measuring an atmospheric pressure. For example, the analysis may be performed in accordance with the following simple method. Here, a process at a certain spot will be focused on in the description.

First, the processing unit 41 of the main server 4 transmits information regarding a time of the spot to the weather server 5 based on the time and positional coordinates (latitude and longitude) included in measurement data of the spot. The processing unit 51 of the weather server 5 reads the information (temperature and the like) of the weather of the spots at the time from the storage unit 54 and transmits the information to the main server 4. The processing unit 41 of the main server 4 receives the temperature from the weather server 5.

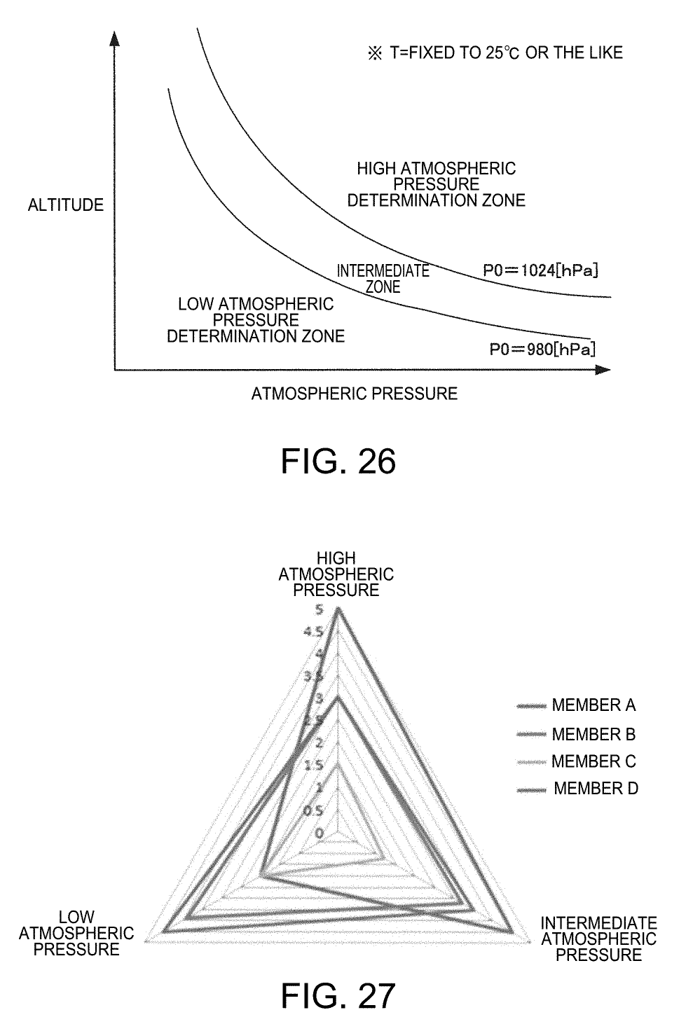

The processing unit 41 of the main server 4 acquires a graph (see FIG. 26) including an approximate curve of a relation between atmospheric pressure and altitude. The graph is a graph or the like stored in advance for each temperature in the storage unit 44.

The processing unit 41 of the main server 4 acquires an altitude of the spot (for example, an altitude which is based on an output of the GPS sensor 110 or an altitude which is based on topographic data), plots the altitude and the atmospheric pressure of the spot on the graph, and determines the class to which the spot belongs (that is, the class to which the measurement data of the spot belongs) in accordance with a plot destination. In the example of FIG. 26, when the plot destination is on the lower left side of the sheet surface, “low atmospheric pressure” is determined. When the plot destination is on the upper right side of the sheet surface, “high atmospheric pressure” is determined.

That is, the processing unit 41 can analyze a change in the physical ability by atmospheric pressure by performing the foregoing clustering on the measurement data of the spots and calculating the indexes for each class. Accordingly, the user can grasp his or her physical ability by atmospheric pressure of the user, such as “strong to low atmospheric pressure” or “strong to high atmospheric pressure”.

When the processing unit 41 performs the clustering by weather, as described above, the processing unit 41 can display the index at each atmospheric pressure as a radar chart illustrated in, for example, FIG. 27. The index is, for example, one of the “stamina index”, the “gear index”, and the “slope ground adaptability index”. A display destination of the radar chart is the display unit 25 of the information terminal 2, the display unit 170 of the electronic apparatus 1, or the like.

The example illustrated in FIG. 27 is an example in which the index is displayed for each user or each atmospheric pressure. The plurality of users illustrated in FIG. 27 may be a plurality of users who belong to the same mountaineering party or may be a plurality of users who do not belong to the same mountaineering party. A route which is a statistical target of the processing unit 41 may be a single mountaineering route or a route group of various different mountaineering routes.

In the system according to the embodiment, the atmospheric pressure which is based on an output of the pressure sensor 112 or the weather information supplied from the weather server 5 has been used to perform the clustering by weather. However, when a solar panel is installed in the electronic apparatus 1, a sunshine duration, a sunshine intensity, or the like may be measured based on a power generation amount of the solar panel and weather information may be generated based on a result of the measurement. Data of the sunshine duration, the sunshine intensity, or the like is assumed to be read as one piece of measurement data by the information terminal 2 from the electronic apparatus 1 and to be further uploaded to the main server 4. The processing unit 41 of the main server 4 can evaluate the physical ability of the user related to sunshine, such as strong to sunshine” “weak to sunshine”, “strong to cloudy sky, or “weak to cloudy sky” by performing the clustering based on the sunshine time or the sunshine intensity collected in this way.

### 1-9-19. Number of Axes of Radar Chart

As described above, in the system according to the embodiment, the processing unit 41 of the main server 4 displays the mutually different indexes as the radar charts (see FIGS. 13 and 27 and the like). Here, a radar chart is a graph in which balance of three or more mutually different amounts (here, indexes) are represented by points on three or more axes disposed radially and the balance of three or more amounts (here, the indexes) is expressed with a polygon by connecting the points on the axes in straight lines. Accordingly, when the number of indexes is “N”, the number of angles of the polygon is also “N”.

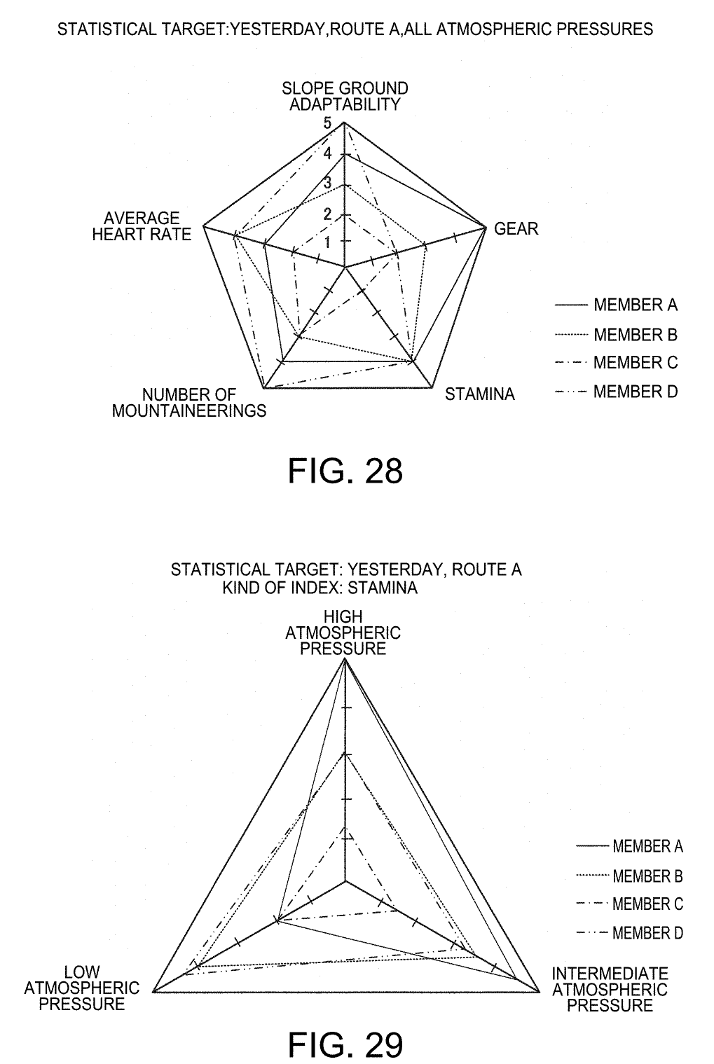

As illustrated in FIG. 28, the processing unit 41 of the main server 4 can also display other indexes as a radar chart along with the stamina index, the gear index, and the slope ground adaptability index. In the radar chart illustrated in FIG. 28, an average heart rate and the number of mountaineerings (an experimental value of the mountaineering route) are represented by mutually different axes in addition to the stamina index, the gear index, and the slope ground adaptability index.

Accordingly, the processing unit 41 of the main server 4 according to the embodiment can select various indexes as calculation targets and display balance of the indexes as a radar chart, for example, in response to an instruction from the user. A display destination of the radar chart is the display unit 25 of the information terminal 2 or the like, as in the above-described example. For example, the instruction from the user is input from the operation unit 23 of the information terminal 2 and is transmitted to the main server 4 via the information terminal 2 and the network 3.

Incidentally, in the radar chart illustrated in FIG. 28, since the number of indexes is “5”, the number of angles of the polygon indicating balance of the indexes is “5”. Since the radar chart illustrated in FIG. 28, data (polygon) related to the plurality of users are superimposed and displayed. Therefore, it is possible to view not only the balance of the indexes but also a variation between the users.

### 1-9-20. Statistical Target

The processing unit 41 of the main server 4 may display a statistical target, a type of index or the like adopted at the time of calculation of the indexes along with the radar chart, as illustrated on the upper side of the sheet surface of FIGS. 28 and 29 or the like.

The “statistical target” mentioned here is a range to which the measurement data used to calculate the indexes belongs and can also be said to be an “index calculation condition”, a “statistical range”, or a “population”. The “type of index” is information indicating that the calculated index is one of the stamina index, the gear index, the slope ground adaptability index, and the like.

The processing unit 41 of the main server 4 according to the embodiment can change a combination of the statistical target and the type of index variously in response to an instruction from the user.

For example, the processing unit 41 of the main server 4 can set “only an uphill ground”, “only a downhill ground”, “only a flat ground”, “only a route (section) in which atmospheric pressure is low when the user has moved”, “only a route (section) in which atmospheric pressure is intermediate when the user has moved”, “only a route (section) in which atmospheric pressure is high when the user has moved”, “only the year of 2015”, “only the year of 2014”, “only the year of 2013”, “only winter season”, “only summer season” “only spring and fall seasons”, or the like as the statistical target or can set “only a specific route”, “a specific route for a specific period”, or the like as the statistical target. That is, the processing unit 41 of the main server 4 can temporarily limit the statistical target or spatially limit the statistical target in response to an instruction or the like from the user.

Then, the processing unit 41 of the main server 4 displays the actually adopted statistical target or kind of indexes or the like as follows.

For example, the processing unit 41 of the main server 4 displays text image “yesterday”, “route A”, and “all atmospheric pressures” indicating the statistical target when the indexes are calculated based on the measurement data of all the sections of a specific mountaineering route on a specific date (for example, the mountaineering route A yesterday) irrespective of an uphill ground, a flat ground, and a downhill ground, the atmospheric pressure, as illustrated in FIG. 28.

The processing unit 41 of the main server 4 displays the text image “yesterday” and “route A” indicating the statistical target and a text image “stamina” indicating the type of index at the time of displaying a radar chart expressing a specific index (for example, the stamina index) related to a spot of a high atmospheric pressure, the index related to a spot of an intermediate atmospheric pressure, and the index related to a spot of a low atmospheric pressure irrespective of an uphill ground, a flat ground, and a downhill ground on mutually different axes, as illustrated in FIG. 29.

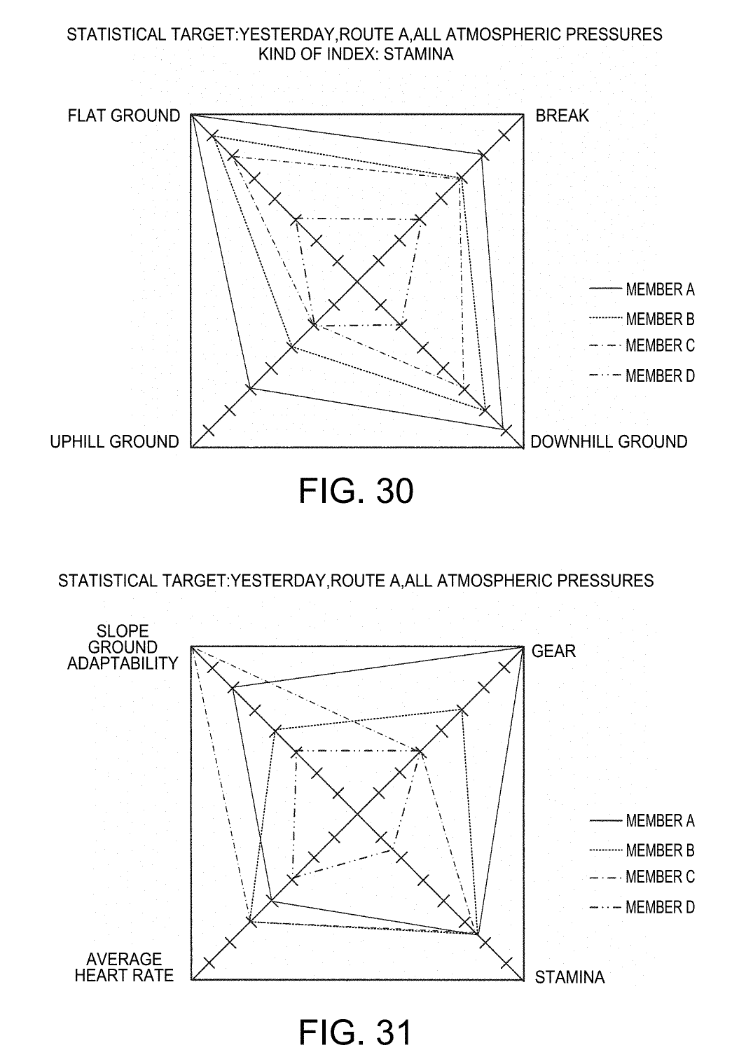

The processing unit 41 of the main server 4 displays the text image “yesterday”, “route A”, and “all atmospheric pressures” indicating the statistical target and a text image “stamina” indicating the type of index at the time of displaying a radar chart expressing a specific index (for example, the stamina index) related to a spot of a flat ground, the index related to a break, and the index related to a downhill ground, and the index related to an uphill ground irrespective of an atmospheric pressure on mutually different axes, as illustrated in FIG. 30.

The processing unit 41 of the main server 4 displays the text image “yesterday”, “route A”, and “all atmospheric pressures” indicating the statistical target at the time of displaying a radar chart expressing the stamina index, the slope ground adaptability index, the gear index, and the average heart rate related to a specific mountaineering route (for example, the mountaineering route A of yesterday) on a specific date, irrespective of an atmospheric pressure on mutually different axes, as illustrated in FIG. 31.

As described above, the processing unit 41 of the main server 4 can calculate the various indexes and change the statistical target variously. Then, the processing unit 41 of the main server 4 can display the calculated three or more indexes as the radar charts, for example, as illustrated in FIGS. 13, 27, and 28 to 31. According to the radar charts are effective as assistances with which a user can intuitively grasp the balance of the plurality of indexes related to the same user or a difference in the balance between the users.

### 1-9-21. Result Display Screen

In the system according to the embodiment, the output unit may output a diagnosis result for each of the plurality of users based on the balance of the physical abilities of each of the plurality of users. In this case, it is possible to objectively and fairly evaluate the plurality of users. Hereinafter, specific description will be made.

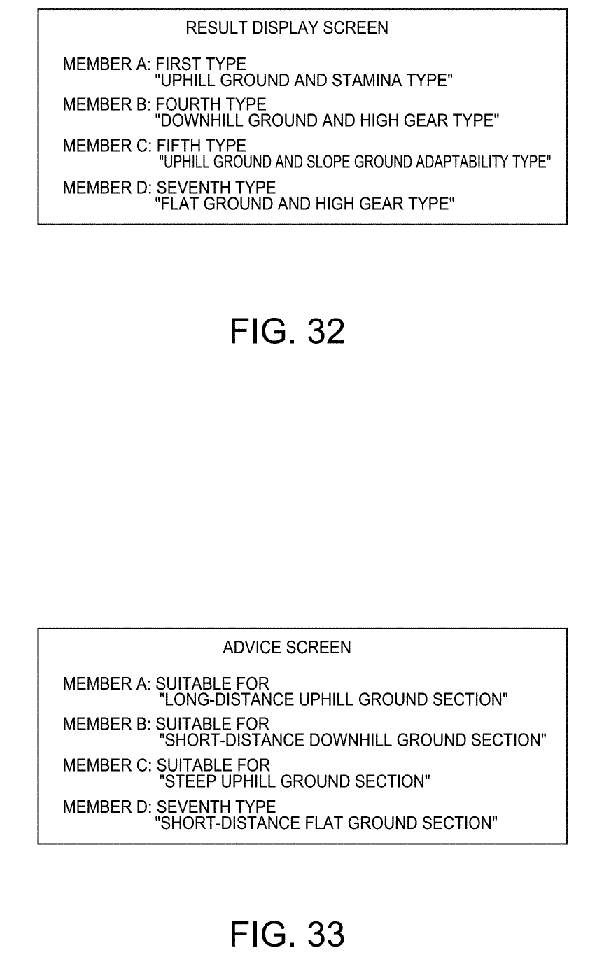

For example, the processing unit 41 of the main server 4 may, for example, a result display screen illustrated in FIG. 32 along with the radar chart or instead of the radar chart. A display destination of the result display screen is the display unit 25 of the information terminal 2 or the like, as in the above-described example.

A text image indicating each type of user is disposed on the result display screen. Here, an example in which the type of user is diagnosed assuming that the number of users is “4” and using eight types of indexes (first to eighth indexes) will be described. Four users (members A, B, C, and D) are members of the same mountaineering party and the measurement data related to a specific mountaineering route for a certain specific period is set as a statistical target.

First, the processing unit 41 of the main server 4 calculates the following first to eighth indexes for each of the four users (members A, B, C, and D).

(i) the first index: the stamina index related to the uphill ground

(ii) the second index: the stamina index related to the downhill ground

(iii) the third index: the gear index related to the uphill ground

(iv) the fourth index: the gear index related to the downhill ground

(v) the fifth index: the slope ground adaptability index related to the uphill ground

(vi) the sixth index: the slope ground adaptability index related to the downhill ground

(vii) the seventh index: the gear index related to the flat ground

(viii) the eighth index: the stamina index related to the flat ground

Next, the processing unit 41 of the main server 4 ranks the four users (members A, B, C, and D) in accordance with the first index. Here, a higher rank is assigned to the user who has the higher physical ability related to the first index.

The processing unit 41 of the main server 4 ranks the four users (members A, B, C, and D) in accordance with the second index. Here, a higher rank is assigned to the user who has the higher physical ability related to the second index.

The processing unit 41 of the main server 4 ranks the four users (members A, B, C, and D) in accordance with the third index. Here, a higher rank is assigned to the user who has the higher physical ability related to the third index.

The processing unit 41 of the main server 4 ranks the four users (members A, B, C, and D) in accordance with the fourth index. Here, a higher rank is assigned to the user who has the higher physical ability related to the fourth index.

The processing unit 41 of the main server 4 ranks the four users (members A, B, C, and D) in accordance with the fifth index. Here, a higher rank is assigned to the user who has the higher physical ability related to the fifth index.

The processing unit 41 of the main server 4 ranks the four users (members A, B, C, and D) in accordance with the sixth index. Here, a higher rank is assigned to the user who has the higher physical ability related to the sixth index.

The processing unit 41 of the main server 4 ranks the four users (members A, B, C, and D) in accordance with the seventh index. Here, a higher rank is assigned to the user who has the higher physical ability related to the seventh index.

The processing unit 41 of the main server 4 ranks the four users (members A, B, C, and D) in accordance with the eighth index. Here, a higher rank is assigned to the user who has the higher physical ability related to the eighth index.

Subsequently, the processing unit 41 of the main server 4 compares the ranks of member A in regard to the first to eighth indexes and diagnoses the type corresponding to the highest index as the type of member A. For example, when the highest index is the first index, the type of member A is diagnosed as a “first type (uphill ground and stamina type)”.

Subsequently, the processing unit 41 of the main server 4 compares the ranks of member B in regard to the first to eighth indexes and diagnoses the type corresponding to the highest index as the type of member B. For example, when the highest index is the fourth index, the type of member B is diagnosed as a “fourth type (downhill ground and high gear type)”.

Subsequently, the processing unit 41 of the main server 4 compares the ranks of member C in regard to the first to eighth indexes and diagnoses the type corresponding to the highest index as the type of member C. For example, when the highest index is the fifth index, the type of member C is diagnosed as a “fifth type (uphill ground and slope ground adaptability type)”.

Subsequently, the processing unit 41 of the main server 4 compares the ranks of member D in regard to the first to eighth indexes and diagnoses the type corresponding to the highest index as the type of member D. For example, when the highest index is the seventh index, the type of member D is diagnosed as a “seventh type (flat ground and high gear type)”.

Further, the processing unit 41 of the main server 4 displays a text image indicating the diagnosed type for each user, for example, as illustrated in FIG. 32. It is possible to express the type using an icon or the like instead of the image type. For example, names of the types can be set as follows.

(i) a first type: an “uphill ground and stamina type”

(ii) a second type: a “downhill ground and stamina type”

(iii) a third type: “uphill ground and high gear type”

(iv) a fourth type: “downhill ground and high gear type”

(v) a fifth type: an “uphill ground and slope ground adaptability type”

(vi) a sixth type: a “downhill ground and slope ground adaptability type”

(vii) a seventh type: a “flat ground and high gear type”

(viii) an eighth type: a “flat ground and stamina type”

As described above, in the system according to the embodiment, the diagnosis result may include the physical ability types (the first to eighth types) of users or may include advice (suitable route type) for the user in addition to the physical ability types or instead of the physical ability types. In this case, it is easy to plan content (route selection, role-sharing, and the like) of exercises of the plurality of users or achieve an improvement in safety or performance of all the plurality of users. Hereinafter, specific description will be made.

The processing unit 41 of the main server 4 can display advice for each type, for example, as illustrated in FIG. 33 instead of displaying each type of user or by displaying each type of user together. In the example illustrated in FIG. 33, types of mountaineering routes (section) suitable for the users are displayed as advice for the users. For example, the advice for each type can be set as follows.

(i) an “uphill ground and stamina type”: “suitable for a long-distance uphill ground section”

(ii) a “downhill ground and stamina type”: “suitable for a long-distance downhill ground section”

(iii) an “uphill ground and high gear type”: “suitable for a short-distance uphill ground section”

(iv) a “downhill ground and high gear type”: “suitable for a short-distance downhill ground section”

(v) an “uphill ground and slope ground adaptability type”: “suitable for a step uphill ground section”

(vi) a “downhill ground and slope ground adaptability type”: “suitable for a steep downhill ground section”

(vii) a “flat ground and high gear type”: “suitable for a short-distance flat ground section”

(viii) a “flat ground and stamina type”: “suitable for a long-distance flat ground section”

According to the advice, each user can objectively grasp the section or the type of route suitable for the user.

### 1-9-22. Display Destination of Screen

As described above, a display destination of a radar chart, a result display screen, or an advice screen related to a plurality of users belonging to the same mountaineering party or sports team may be, for example, at least one of the information terminals 2 used by the plurality of users or may be at least one of the electronic apparatuses 1 used by the plurality of users. For example, the display destination may be the information terminal 2 or the electronic apparatus 1 used by a representative (leader) of the plurality of users.

### 1-9-23. Information Terminal of Coach

In the foregoing embodiment, a display destination of a radar chart, a result display screen, or an advice screen related to a plurality of users belonging to the same mountaineering party or sports team may be the information terminal 2 or the electronic apparatus 1 used by a manager (coach) of the plurality of users.

For example, when the display destination is set to the information terminal 2 or the electronic apparatus 1 of a coach of a long-distance race team, it is easy for the coach to determine role-sharing (assignment of long-distance race sections) of users (players) of the long-distance race team or select an individual training menu of the users (players) of the long-distance race team based on at least one of the radar chart, the type, and the advice displayed on the information terminal 2 or the electronic apparatus 1.

### 1-9-24. Automatic Assignment

In the system according to the embodiment, when a correspondent relation between sections of a mountaineering route (long-distance race route) and types of users suitable for the sections is recorded in advance in the storage unit 44 of the main server 4, the processing unit 41 of the main server 4 can also assign the players to the sections (assign the player for each section) instead of the coach.

For example, the coach of the long-distance race team inputs a correspondent relation between sections of a predetermined mountaineering route (long-distance race route) and types suitable for the sections in advance through the operation unit 23 of the information terminal 2 used by the coach. Information regarding the input correspondent relation is transmitted to the main server 4 via the information terminal 2 and the network 3.

When the information regarding the correspondent relation is received, the processing unit 41 of the main server 4 records the information regarding the correspondent relation in association with data of the corresponding mountaineering route (long-distance race route) in the storage unit 44. Then, when the type related to the mountaineering route is diagnosed for each of the players of the long-distance race team, the processing unit 41 of the main server 4 determines to assign each section to each player based on the type which is a diagnosis result of each player and the correspondent relation in the storage unit 44 and transmits information of the determined assignment to the information terminal 2 of the coach via the network 3. The processing unit 21 of the information terminal 2 displays the received information of the assignment on the display unit 25.

Accordingly, the coach can simply assign the plurality of users (players) to the sections of the long-distance race route.

### 1-10. Operational Effects of Embodiment

The system according to the embodiment includes the output unit that outputs the plurality of indexes related to the heart beat information of a user as information related to the balance of the physical abilities of the user. In the related art, the user merely experimentally grasped to which type the user is relevant among various types such as a type strong in a long distance, a type strong to a short distance, a type strong to an uphill ground, a type strong to a downhill ground, and a type strong to a flat ground. However, according to the information (that is, the information related to the balance of the physical abilities) which is based on the plurality of indexes related to the heart beat information, the user can objectively grasp his or her physical ability type.

Since the system according to the embodiment can cluster the measurement data and calculate the indexes for each class, it is also possible to evaluate the physical ability of the user by situations, such as “strong to an uphill ground”, “strong to a flat ground”, “strong to a downhill ground”, “strong to a low atmospheric pressure”, “strong to a high atmospheric pressure”, “strong to long-time sunshine”, and “strong to strong sunshine”.

The system according to the embodiment can give a user a motivation for mountaineering through an observation of a change in the physical ability by visualizing the indexes with numerical values or the like or gives a user a pleasure for mountaineering with an amusement feeling through a change in the physical ability.

In the system according to the embodiment, a change in a condition can also be grasped from the change in the physical ability. Therefore, by performing feedback such as pace-building of a subsequent mountaineering plan, it is easy to manage a long-term pace. Thus, it is possible to expect the advantage of avoiding a danger or preventing a disaster due to an unreasonable plan.

In the system according to the embodiment, a graph (radar chart) related to the plurality of indexes is displayed. A radar chart is a graph in which three or more mutually different amounts (here, the indexes) are represented by points on three or more axes disposed radially and the balance of three or more amounts (here, the indexes) is expressed with a polygon by connecting the points on the axes in straight lines. Accordingly, it is easy for the user to intuitively grasp the balance of his or her physical ability using the shape of the polygon.

### 2. Modification Examples

### 2-1. Assigning Comment

In the system according to the embodiment, the storage unit 44 of the server 4 may store a comment in association with route information and heart beat information associated with each other. For example, the processing unit 41 writes the comment and a writing destination of the comment is, for example, a file of a mountaineering route. The comment is a comment input by the user. For example, the user inputs the comment to the server 4 through an application program (a web app) installed in advance in the information terminal 2 such as a smartphone or the electronic apparatus 1 such as an outdoor watch.

The comment input by the user includes, for example, an impression (hard, easy, or the like) when the user thinks back on the exercise (mountaineering, running, or the like) related to the mountaineering route or a memory (pleasant, boring, or the like).

Accordingly, when the storage unit 44 associates the comment with the route information and the heart beat information, the processing unit 41 can allow relevance between the impression or the memory of the user and the actual heart beat information or relevance between the impression or the memory of the user and the plurality of indexes.

### 2-2. Using Database of Another User

In the system according to the foregoing embodiment, the range which is a statistical target is limited within a range of the database of an individual user, but the range may be broadened to various databases of users. That is, the indexes may be calculated as statistical data of a team, the indexes may be calculated as statistical data of a region, or the indexes may be calculated as statistical data of friends.

### 2-3. Continuity of Data

The case in which only one user uses only one electronic apparatus 1 in the system according to the foregoing embodiment has been exemplified, but one user may use the plurality of electronic apparatuses 1. In this case, the main server 4 may manage the plurality of electronic apparatuses 1 in association with one user. For example, since this structure enables the database to continue when the user exchanges the electronic apparatus 1, the structure is convenient.

### 2-4. Map Display of Index or the Like

In the system according to the foregoing embodiment, at least one of the heart beat and the index related to a mountaineering route may be displayed along with a map of the mountaineering route. A display destination of the map or the like is, for example, the display unit 25 of the information terminal 2. For example, when the user designates a certain spot of the mountaineering route (for example, a mountaineering route indicated by a curve image) displayed on a map, at least one of the heart beat and the index related to the spot may be displayed in a pop-up manner. A display destination of the map or the like is the display unit 25 of the information terminal 2, the display unit 170 of the electronic apparatus 1, or the like.

For example, when the spot designated by the user is an “uphill ground”, the index of the uphill ground is displayed in a pop-up manner. When the spot designated by the user is a “downhill ground”, the index of the downhill ground is displayed in a pop-up manner. When the spot designated by the user is a “flat ground”, the index of the flat ground is displayed in a pop-up manner.

For example, when the spot designated by the user is an “a spot high at altitude”, the index of the spot high at altitude is displayed in a pop-up manner. When the spot designated by the user is an “a spot low at altitude”, the index of the spot low at altitude is displayed in a pop-up manner.

The “index at the spot high at altitude” mentioned here is an index calculated setting measurement data at the spot of which an altitude is equal to or greater than a threshold as a statistical target. The “index at the spot low at altitude” mentioned here is an index calculated setting measurement data at the spot of which an altitude is less than the threshold as a statistical target. When the index is calculated, the measurement data may be clustered in accordance with the altitude.

### 3. Other Modification Examples

The invention is not limited to the foregoing embodiment, but various modifications can be made within the scope of the gist of the invention.

For example, in the foregoing embodiment, some or all of the functions of the main server 4 may be mounted on the information terminal 2 or the electronic apparatus 1, some of the functions of the information terminal 2 and the electronic apparatus 1 may be mounted on the main server 4, some or all of the functions of the electronic apparatus 1 may be mounted on the information terminal 2, or some or all of the functions of the information terminal 2 may be mounted on the electronic apparatus 1.

The functions of a known smartphone, for example, a camera function, a calling function, and a communication function, may be mounted on the electronic apparatus 1 or the information terminal 2.

Another sensing function (a humidity sensor or the like) of sensing at least some of sport activities (including biological activities) may be mounted on the electronic apparatus 1 or the information terminal 2.

The electronic apparatus 1 or the information terminal 2 can be configured with any of various types of portable information apparatuses such as a wrist type electronic apparatus, an earphone type electronic apparatus, a ring type electronic apparatus, a pendant type electronic apparatus, an electronic apparatus worn on a sports instrument, a smartphone, and a head-mounted display (HMD).

The electronic apparatus 1 or the information terminal 2 may notify of information regarding the user through image display, sound output, vibration, and the like or in a combination of at least two of the image display, the sound output, the vibration.

In the foregoing embodiment, a Global Positioning System (GPS) has been used as a satellite positioning system, but another global navigation satellite system (GNSS) may be used. For example, one or two or more of satellite positioning systems such as European Geostationary-Satellite Navigation Overlay Service (EGNOS), Quasi Zenith Satellite System (QZSS), GLObal NAvigation Satellite System (GLONASS), GALILEO, and BeiDou Navigation Satellite System (BeiDou) may be used. A satellite-based augmentation system (SBAS) such as a wide area augmentation system (WAAS) may be used for at least one of the satellite positioning systems.

The foregoing embodiments and modification examples are merely examples, but the invention is not limited thereto. For example, the embodiments and the modification examples can also be appropriately combined.

The invention includes substantially the same configurations (for example, configurations in which functions, methods, and results are the same or configurations in which objectives and effects are the same) as the configurations described in the embodiments. The invention includes configurations in which unsubstantial portions of the configurations described in the embodiment are replaced. The invention includes configurations in which the same operational effects as the configurations described above or configurations in which the same objectives can be achieved. The invention includes configurations in which known technologies are added to the configuration described in the embodiments.

What is claimed is:

1. A physical ability evaluation system comprising:
an output unit that outputs a plurality of indexes related to heart beat information of a user as information related to balance of a physical ability of the user.

2. The physical ability evaluation system according to claim 1,
wherein the plurality of indexes include at least one of an index indicating a degree of a change in the heart beat information with respect to an elapsed time of the user, an index indicating a degree of a change in the heart beat information with respect to a change in a speed of the user in a direction perpendicular to a gravity direction, and an index indicating a degree of a change in the heart beat information with respect to a change in a speed of the user in the gravity direction.

3. The physical ability evaluation system according to claim 1,
wherein the plurality of indexes include at least one of a plurality of indexes calculated for each period in which the user exercises, a plurality of indexes calculated for each weather of a route along which the user exercises, and a plurality of indexes calculated for each gradient of the route along which the user exercises.

4. The physical ability evaluation system according to claim 1,
wherein the output unit outputs a graph related to the plurality of indexes.

5. The physical ability evaluation system according to claim 4,
wherein the number of users is plural, and
wherein the output unit outputs the graph related to the plurality of users.

6. The physical ability evaluation system according to claim 5,
wherein the plurality of users are users belonging to the same group.

7. The physical ability evaluation system according to claim 5,
wherein the output unit outputs a diagnosis result for each plurality of users.

8. The physical ability evaluation system according to claim 7,
wherein the diagnosis result includes at least one of a physical ability type of the user and advice for the user.

9. The physical ability evaluation system according to claim 1, further comprising:
a storage unit that stores route information of a route along which the user exercises and the heart beat information in association with each other.

10. An electronic apparatus comprising:
an output unit that outputs a plurality of indexes related to heart beat information of a user as information related to balance of a physical ability of the user.

11. A physical ability evaluation server comprising:
an output unit that outputs a plurality of indexes related to heart beat information of a user as information related to balance of a physical ability of the user.

12. A physical ability evaluation method comprising:
outputting a plurality of indexes related to heart beat information of a user as information related to balance of a physical ability of the user.

13. The physical ability evaluation method according to claim 12,
wherein the plurality of indexes include at least one of an index indicating a degree of a change in the heart beat information with respect to an elapsed time of the user, an index indicating a degree of a change in the heart beat information with respect to a change in a speed of the user in a direction perpendicular to a gravity direction, and an index indicating a degree of a change in the heart beat information with respect to a change in a speed of the user in the gravity direction.

14. The physical ability evaluation method according to claim 12,
wherein the plurality of indexes include at least one of a plurality of indexes calculated for each period in which the user exercises, a plurality of indexes calculated for each weather of a route along which the user exercises, and a plurality of indexes calculated for each gradient of the route along which the user exercises.

15. The physical ability evaluation method according to claim 12,
wherein in the outputting of the plurality of indexes, a graph related to the plurality of indexes is output.

16. The physical ability evaluation method according to claim 15,
wherein the number of users is plural, and
wherein in the outputting of the plurality of indexes, the graph related to the indexes of the plurality of users is output.

17. The physical ability evaluation method according to claim 16,
wherein the plurality of users are users belonging to the same group.

18. The physical ability evaluation method according to claim 16,
wherein in the outputting of the plurality of indexes, a diagnosis result for each plurality of users is output.

19. The physical ability evaluation method according to claim 18,
wherein the diagnosis result includes at least one of a physical ability type of the user and advice for the user.

20. A recording medium that records a program causing a computer to perform:
outputting a plurality of indexes related to heart beat information of a user as information related to balance of a physical ability of the user.

21. An electronic apparatus outputting a plurality of indexes related to heart beat information of a user as information related to balance of a physical ability of the user.

22. The electronic apparatus according to claim 21,
wherein the plurality of indexes include at least one of an index indicating a degree of a change in the heart beat information with respect to an elapsed time of the user, an index indicating a degree of a change in the heart beat information with respect to a change in a speed of the user in a horizontal direction perpendicular to a gravity direction, and an index indicating a degree of a change in the heart beat information with respect to a change in a speed of the user in the gravity direction.

23. The electronic apparatus according to claim 21,
wherein the plurality of indexes include at least one of a plurality of indexes calculated for each period in which the user exercises, a plurality of indexes calculated for each weather of a route along which the user exercises, and a plurality of indexes calculated for each gradient of the route along which the user exercises.

24. The electronic apparatus according to claim 21,
wherein a graph related to the plurality of indexes is output.

25. The electronic apparatus according to claim 24,
wherein the number of users is plural, and
wherein the graph related to the plurality of users is output.

26. The electronic apparatus according to claim 25,
wherein the plurality of users are users belonging to the same group.

27. The electronic apparatus according to claim 25,
wherein a diagnosis result for each plurality of users is output.

28. The electronic apparatus according to claim 27,
wherein the diagnosis result includes at least one of a physical ability type of the user and advice for the user.

29. The electronic apparatus according to claim 21,
wherein route information of a route along which the user exercises is stored in association with the heart beat information.

30. The physical ability evaluation system according to claim 9,
wherein the storage unit stores a comment in association with the associated route information and heart beat information.

31. The electronic apparatus according to claim 29,
wherein a comment is stored in association with the associated route information and heart beat information.
